(19) United States
(12) Patent Application Publication
(10) Pub. No.: US 2026/0283719 A1
(43) Pub. Date: Sep. 24, 2026

(54) SYSTEM AND METHOD FOR AN ACTIVE CANNULA

(71) Applicants: The Brigham and Women's Hospital, Inc., Boston, MA (US); The General Hospital Corporation, Boston, MA (US); The University of Western Ontario, London (CA)

(72) Inventors: Jayender Jagadeesan, Boston, MA (US); Ruisi Zhang, Boston, MA (US); Rajnikant V. Patel, London (CA); Dianne E. Sacco, Boston, MA (US); Filipe Pedrosa, London (CA); Navid Feizi, London (CA)

(21) Appl. No.: 19/477,064

(22) PCT Filed: Apr. 19, 2024
(86) PCT No.: PCT/US2024/025570

§ 371 Date: Oct. 20, 2025

Related U.S. Application Data
(60) Provisional application No. 63/460,461, filed on Apr. 19, 2023.

(51) Int. Cl. A61B 34/30; A61B 17/00; A61B 17/34; A61B 34/10; A61B 34/20; A61B 90/00; G06T 7/00; G06T 7/11

(52) U.S. Cl. CPC A61B 34/30; A61B 17/3421; A61B 34/10; A61B 34/20; A61B 90/06; G06T 7/0012; G06T 7/11; A61B 2017/00022; A61B 2017/00398; A61B 2017/00477; A61B 2017/00867; A61B 2034/105; A61B 2034/2051; A61B 2090/064; G06T 2207/10081; G06T 2207/10088

(57) ABSTRACT

An active cannula robot system is provided. The system can include a housing with a proximal end and a distal end. The housing can surround a cannula unit, including two motors positioned proximally in the housing. The system can also include a carriage unit operably coupled and positioned distally to the two motors within the housing. The system can also include an active cannula tube extending longitudinally between and beyond the proximal end and distal end and configured to be actuated by the carriage unit. Also, the system can include a stationary cannula tube fixed to an outer surface of the distal end of the housing and extending distally and a processor configured to control the active cannula robot system.

### CROSS REFERENCE TO RELATED APPLICATIONS

The present application is based on, claims priority to, and incorporates herein by reference in its entirety for all purposes, U.S. Provisional Application Ser. No. 63/460,461, filed Apr. 19, 2023.

### STATEMENT OF GOVERNMENT SUPPORT

This invention was made with government support under 5R01DK119269-05 awarded by the National Institutes of Health. The government has certain rights in the invention.

### BACKGROUND

The present disclosure relates to interventional medical devices and, more particularly, to systems and methods that utilize an active cannula for accessing a surgical site through small incisions or natural orifices.

Minimally invasive surgery reduces the trauma associated with traditional open surgery, resulting in faster recovery time, fewer wound infections, reduced postoperative pain, and improved cosmesis. Steerable devices are often used in minimally invasive surgery to improve a surgeon's dexterity inside the patient as a means of safely navigating internal structures. One known steerable device includes multiple joints and tendons at the distal end, and the drive system at the proximal end. However, these steerable devices may be associated with large footprints and complex internal components, making them bulky and difficult for hand-held use.

Other steerable devices may include separate handles for holding and securing the position of the device by the user, wherein the other hand is used to manipulate the minimally invasive surgery device in the surgical field. These types of devices suffer in their degree of accuracy since the user must ensure correct positioning of both hands, and results in user fatigue during a minimally invasive procedure.

One approach to this problem is robotic positioning of the device. This however further increases the footprint of the system in the operating room, increases cost and duration of surgery, and reduces the versatility of the device for different medical environments.

Thus, there is a continuing need for systems and methods for improved interventional medical procedures.

### SUMMARY

The present disclosure addresses the aforementioned drawbacks by providing systems and methods for an active cannula robot (ACR). The ACR may include a small footprint actuation mechanism for providing rotational and/or translational motion to one or more concentric cannula. Thereby, each concentric tube is afforded motion in at least two degrees of freedom (DOF). The structural components of the actuation mechanism can be designed to fit inside a housing configured for single-handed use for improved dexterity and accuracy for biopsy or treatment of a target region inside a patient, while controlling the risk of damage to adjacent tissues.

In one aspect of the present disclosure, an active cannula robot system is provided. The system can include a housing with a proximal end and a distal end. The housing includes a first cannula unit, including a first motor and a second motor positioned proximally in the housing. The system can also include a carriage unit operably coupled and positioned distally to the first motor and the second motor within the housing. The system can also include an active cannula tube extending longitudinally between and beyond the proximal end and distal end and configured to be actuated by the carriage unit. Also, the system can include a stationary cannula tube fixed to an outer surface of the distal end of the housing and extending distally and a processor configured to control the active cannula robot system.

In another aspect of the present disclosure, an active cannula robot system is provided. The system includes a housing with a proximal end and a distal end, wherein the proximal end and distal end are reversibly separable into a proximal portion and a distal portion. The housing includes the proximal portion which includes one or more pairs of motors, and a first coupling operably connected and distally positioned to each motor in each of the one or more pairs of motors. The housing includes the distal portion which includes one or more carriage units corresponding to a number of the one or more pairs of motors, and two second couplings operably connected and proximally positioned to each carriage unit, wherein each of the first couplings and each of the second couplings reversibly connect to provide a mechanical and an electrical connection between each of the one or more pairs of motors and the one or more carriage units. The distal portion further includes a stationary cannula tube fixed to an outer surface of the distal end and extending distally. The distal portion additionally includes a channel coupled to the outer surface of the distal portion. The housing further includes an active cannula tube configured to be actuated by the one or more carriage units and introduced into the distal portion by the channel and extend longitudinally therein and distally outside the distal end. The system further includes a processor configured to control the active cannula robot system.

In another aspect of the present disclosure, a method of intraoperative navigation of an active cannula robot is provided. The method includes the steps of acquiring pre-operative medical imaging data, applying image segmentation to the medical imaging data, developing a three-dimensional model of one or more structures in the segmented medical image data, developing a registration algorithm of an electromagnetic tracking system, wherein the electromagnetic tracking system is configured to monitor a position of a distal tip of the ACR; and, registering the electromagnetic tracking system to the pre-operative medical image data.

The foregoing and other aspects and advantages of the invention will appear from the following description. In the description, reference is made to the accompanying drawings which form a part hereof, and in which there is shown by way of illustration a preferred embodiment of the invention. Such an embodiment does not necessarily represent the full scope of the invention, however, and reference is made therefore to the claims and herein for interpreting the scope of the invention.

### BRIEF DESCRIPTION OF THE DRAWINGS

FIG. 1A is a section view of an active cannula robot (ACR), according to aspects of the present disclosure.

FIG. 1B is a section view of another embodiment of an ACR, according to aspects of the present disclosure.

FIG. 1C is a section view of another embodiment of an ACR, according to aspects of the present disclosure.

FIG. 1D is an alternate view of the ACR of FIG. 1C.

FIG. 1E is an alternate view of the ACR of FIG. 1C.

FIG. 1F is an alternate view of the ACR of FIG. 1C.

FIG. 2A is an exploded view of a tube carriage unit, according to aspects of the present disclosure.

FIG. 2B in another exploded view of the tune carriage unit, according to aspects of the present disclosure.

FIG. 2C is a photograph of the tube carriage unit, according to aspects of the present disclosure.

FIG. 3A shows non-limiting example dimensions of the outer shell of the robot of FIG. 1A and the range of motion of each carriage unit, according to aspects of the present disclosure via a side view.

FIG. 3B shows non-limiting example dimensions of the outer shell of the robot of FIG. 1A via a front view.

FIG. 3C shows a non-limiting example of the dimensions of the outer shell of the robot of FIG. 1B from a front view (left) and side view (right), according to aspects of the present disclosure.

FIG. 3D shows non-limiting example dimensions of the outer shell of the distal end of the robot of FIG. 1C and the range of motion each carriage unit from a side view, according to aspects of the present disclosure.

FIG. 4A shows a photograph of an example hand-held ACR system with four DC motors used to drive the cannula tubes and including three concentric cannula tubes according to aspects of the present disclosure.

FIG. 4B shows a photograph of an example hand-held ACR system and illustrating the detailed structure of an inner cannula tube and a middle tube that has been secured using two carriages, where the linear transition is delivered via a 4 mm diameter lead screw coupled with a flanged nut, and the rotation is delivered via a splined rotary shaft coupled with a linear bearing, which enables four degrees of freedom (4DoF) motion for the hand-held ACR.

FIG. 4C shows a photograph of an example hand-held ACR system wherein an outer tube is stationary and is fixed on a force/torque sensor.

FIG. 5 is a photograph of a stationary tube and two active cannula tubes, according to aspects of the present disclosure.

FIG. 6 is a photograph of the PCB, according to aspects of the present disclosure.

FIG. 7 is a schematic of the active cannula robot (ACR) system, according to aspects of the present disclosure.

FIG. 8A is a photograph of an active cannula robot system using a passive arm, according to aspects of the present disclosure.

FIG. 8B is a photograph of an active cannula robot system operated manually, according to aspects of the present disclosure.

FIG. 9A is a photograph of a single-handed grasp of the active cannula robot, according to aspects of the present disclosure.

FIG. 9B is a photograph of a double-handed grasp of the active cannula robot, according to aspects of the present disclosure.

FIG. 10 is a photograph of a view of the concentric cannula tubes from inside a phantom.

FIG. 11 shows the registration jig used to determine the ACR coordinate frame, according to aspects of the present disclosure.

FIG. 12 shows the steps of a navigation system, according to aspects of the present disclosure.

FIG. 13 is a visualization of a percutaneous nephrolithotomy (PCNL) per-operative plan. A trajectory for delivering the concentric tube robot (CTR) into the collecting system for reaching the renal stones from the skin surface is shown.

FIG. 14A is a comparison between the vertical orientations and handling of the active canula robot and the LithoClast handpiece in a PCNL procedure (PUP: prone upper pole; PMP: supine mid pole; SUP: supine upper pole).

FIG. 14B is a comparison between the horizontal orientations and handling of the active canula robot and the LithoClast handpiece in a PCNL procedure (PUP: prone upper pole; PMP: supine mid pole; SUP: supine upper pole).

FIG. 15 shows plots of normalized mean absolute surface electromyography (EMG) when handling the LithoClast device versus the hand-held active canula robot for prone lower pole (PLP), prone mid pole (PMP), prone upper pole (PUP), supine lower pole (SLP), supine mid pole (SMP), and supine upper pole (SUP) positions. The results shown are for a group of 6 urologists and 5 non-clinicians.

FIG. 16 is a plot of mean-square estimation error as a function of Ge.

FIG. 17 is a schematic of a closed-loop task-spaced position control with an integrated Kalman filter.

FIG. 18A shows plots of ground truth and actual trajectory tracked by the active cannula robot under closed-loop control for the helical, square, and hypocycloidal trajectories. These results were obtained when the robot was attached to a passive arm.

FIG. 18B shows plots of ground truth and actual trajectory tracked by the active cannula robot under closed-loop control for the helical, square, and hypocycloidal trajectories. These results were obtained when the robot was manually grasped by a user.

FIG. 18C shows plots of the hand motion detected by the reference EM sensor attached to the robot.

FIG. 19A shows plots of the ground truth and actual trajectory tracked by the active canula robot under closed-loop control for a PCNL trajectory. These results were obtained when the robot was attached to a passive arm.

FIG. 19B shows plots of the ground truth and actual trajectory tracked by the active canula robot under closed-loop control for a PCNL trajectory. These results were obtained when the robot was manually grasped by a user.

FIG. 19C The diagram on the right shows the hand motion detected by the reference EM sensor attached to the robot.

FIG. 20 is a plot of the distribution of position error under closed-loop control for the helical, square, hypocycloidal, and PCNL trajectories for both the passive arm-supported and hand-grasped scenarios.

### DETAILED DESCRIPTION

A conventional needle does not have the capability of steering within the anatomy once it has been inserted by a few centimeters into the body. Improper placement of the needle could lead to inconclusive results for biopsy or suboptimal delivery of therapy. Further, due to the difficulty in steering the needle, avoiding critical structures such as blood vessels may require multiple insertions and retractions of the needle, leading to greater tissue damage.

Continuum robots are distinguished by their unique continuously bending structures allowing their end effectors to maneuver through complex and constrained environments. This category of robots has gained significant attention in minimally invasive surgery (MIS) due to their small cross-section (less than 1 mm in diameter), and high slenderness (length-to-diameter) ratio, offering a marked difference to standard MIS robots and tools. The miniaturized scale and the end effector dexterity allow these robots to be inserted into the body through small incisions or natural orifices and traverse an anatomical pathway to the target organ. Such capabilities enable the execution of surgical procedures with reduced trauma at the point of entry leading to faster recovery. Furthermore, the natural flexibility of these robots enhances safety features intrinsically.

Continuum robots, designed for MIS, are divided into four main categories: (1) tendon-driven, (2) multi-backbone, (3) magnetically actuated, and (4) concentric tube robots. Tendon-driven robots operate using one or more tendons and are noted for their high flexibility due to the compliance of the tendons. These robots typically consist of a passive section along with an actively bending distal end, enabling navigation through tightly curved paths. This feature makes them particularly useful in cardiovascular procedures, such as ablation. Multi-backbone robots provide enhanced dexterity with a reduced number of actuators by employing push/pull rods. These robots are well suited for procedures that require higher stiffness than what tendon-driven robots can provide, such as for bronchoscopy and abdominal surgery. Magnetically actuated robots are manipulated through an external magnetic field and their insertion is facilitated by a catheter advancer. Lastly, concentric tube robots (CTR), characterized by their higher stiffness than the others and needle-like structure, are particularly effective in tasks requiring tissue interaction with higher impedance, such as puncturing, tissue handling, and drilling.

The present disclosure provides systems and methods for a steerable interventional device that uses concentric tubes which can steer within the anatomy based on the commands of the clinician. The system may be hand-held and/or robotically controlled. The interventional device may be a needle. The tubes may be a memory material. For example, a memory metal may be used, such as nitinol. The systems and methods provided herein may be realized in a variety of different configurations and forms. The terms active cannula robot (ACR) and concentric tube robot (CTR) are used herein to generally describe any or all of these different configurations or implementations. In one non-limiting example, the ACR may include two main parts-concentric tubes and a mechatronic unit, such as will be described.

Further disclosed herein, the navigation system can be used for navigation in three dimensions (3D). As will be described, a method for such 3D navigation may include some setup steps, such as: (1) segmenting and creating 3D models of key anatomical structures from preoperative medical imaging data (such as computed tomography, CT, or magnetic resonance imaging, MRI); (2) registering the electromagnetic tracking system to the preoperative medical imaging data; (3) refining the initial registration using a dynamic registration approach to further refine the registration using implantable sensors or intraoperative imaging like ultrasound imaging, C-arm CT etc.

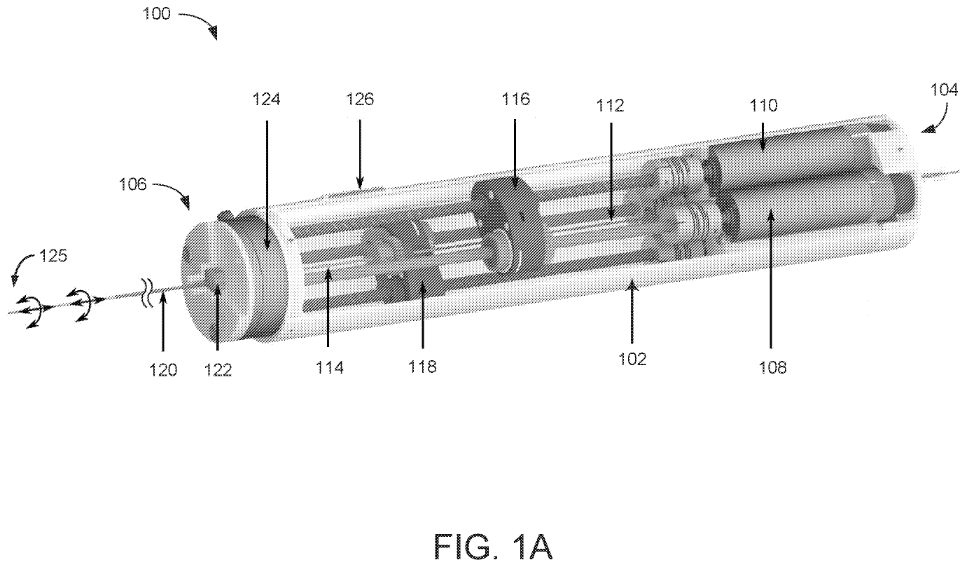

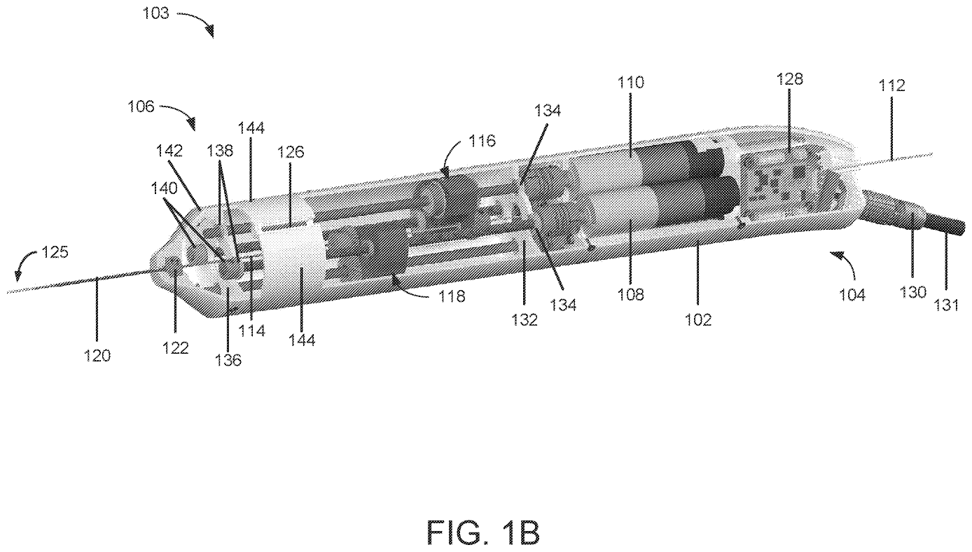

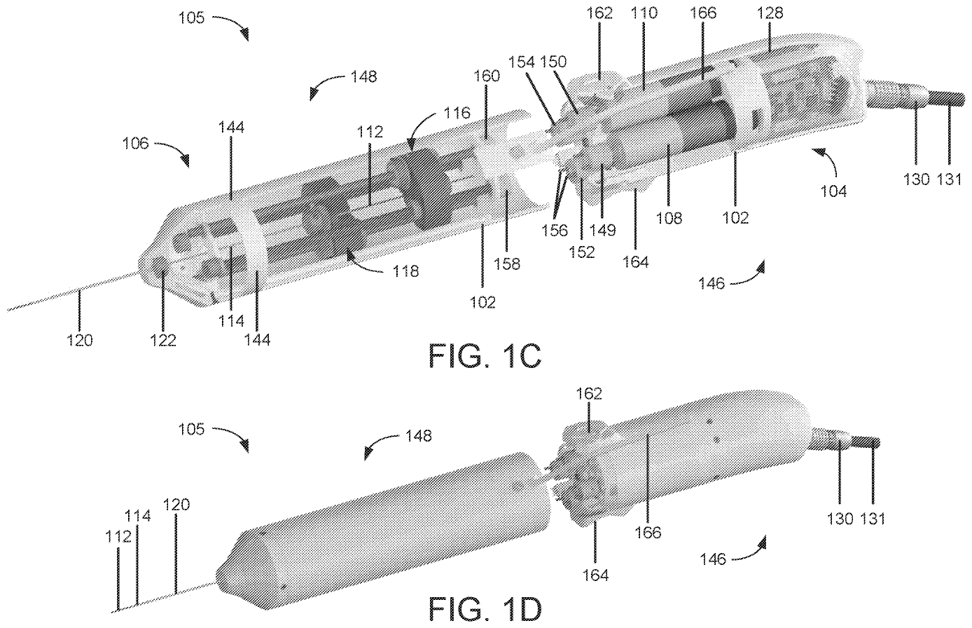

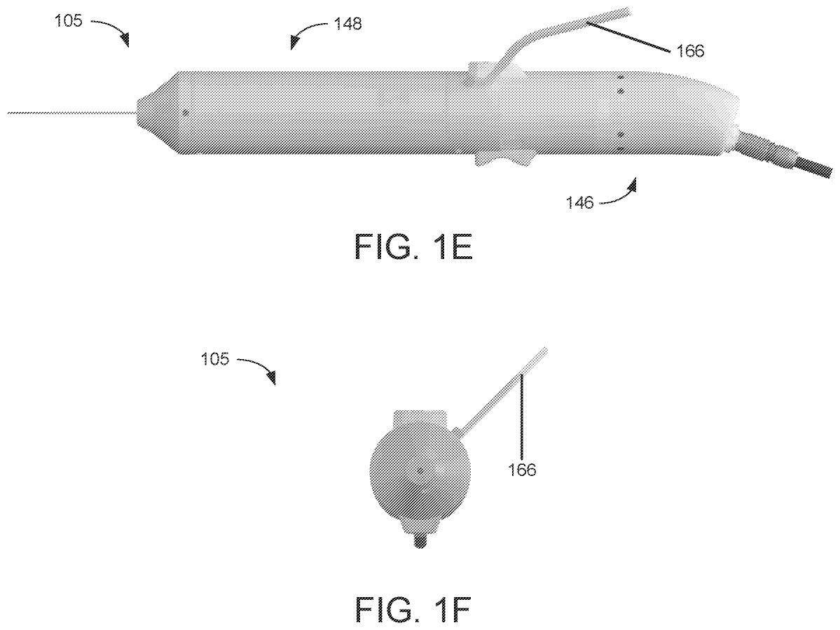

FIG. 1A shows a non-limiting example of the hand-held ACR 100. The ACR 100 includes a housing 102 with a proximal end 104 and a distal end 106. The housing 102 may have a cylindrical outer shell or any ergonomic shape for single-handed grasping and use.

In a non-limiting example, the housing 102 encloses a plurality of components. In the illustrated, non-limiting example, the housing 102 surrounds four motors, two rotation motors 108 and two translation motors 110. Other numbers of motors or other actuator systems may be utilized. In the illustrated, non-limiting example, one of each rotation motors 108 and translation motors 110 are associated with cannula tube 112 and carriage unit 116, or cannula tube 114 and carriage unit 118. In this configuration, each of the rotation motors 108 causes axial rotation of its respective cannula tube 112, 114. Further, in this configuration, each of the translation motors 110 causes a one-dimensional longitudinal motion of its respective cannula tube 112, 114. In a non-limiting example, additional motors, cannula tubes, and carriage units may be included.

According to the illustrated configuration, a first of the cannula tubes 112 is inserted in a second of the cannula tubes 114 in a concentric fashion. In this example, the first cannula tube 112 enters the proximal end 104 of the housing 102, extends through the housing 102, and exits the distal end 106 of the housing 102. Furthermore, as illustrated, the second cannula tube 114 extends partially through the housing 102 and exits the distal end 106 of the housing 102.

In the non-limiting, illustrated configuration, a third cannula tube 120 is provided and can be held by a shaft collar 122 on an outer surface of the distal end 106 of the housing 102. This third cannula tube 120 extends distally from the shaft collar 122 and externally from the housing 102. In this particular configuration, the first and second cannula tubes 112 and 114 are concentrically arranged within the third cannula tube 120. Further, the first cannula tube 112 extends distally from the second cannula tube 114, which itself extends distally from the third cannula tube 120. Thus, in this non-limiting example, the distal tip of first cannula tube 112 corresponds to the distal tip of the ACR system.

In a non-limiting example, diagnostic or therapeutic instruments may be inserted in the innermost cannula for delivery to the target region inside the patient. For example, imaging devices such as fiber optics or ultrasound sensors may be fed through the concentric cannulae. Alternatively, surgical tools for biopsy collection or tissue ablation may be fed through the concentric cannulae. Further, position sensors, such as electromagnetic (EM) sensors may be placed inside the cannulae to track the tip of instrument inside the patient for proper positioning and reduced damage to surrounding tissue.

The ACR 100 may further include a force sensor 124 in the distal end of the housing 102. The ACR system 100 may further include a reference electromagnetic (EM) sensor 126 on the longitudinal outer surface of the housing 102.

In a non-limiting example, the ACR 100 can include a cylindrical outer shell that can be grasped directly by the user, such as using a single hand. Alternatively, the ACR 100 may be fastened to a passive or robotic arm, such as a Kuka robot or da Vinci robot from Intuitive Surgical. This design allows full control without requiring a handle leading to a compact and lightweight design and also provides good manipulability.

The outer shell can enclose some or all components including the motors, actuation units, and sensors. However, in the illustrated configuration, the control units are not similarly enclosed. The ACR 100 can be designed to manipulate multiple tubes that are concentrically combined. In one configuration, the outer tube can be stationary with a straight section. The inner and middle tubes can have multiple degrees of freedom. For example, the inner and middle tubes can have two degrees of freedom (e.g., translation and rotation) and both can include straight and curved sections. In one non-limiting example of a particular implementation, a prototype was constructed that includes tubes with dimensions, such as those listed in Table 1.

TABLE 1
The dimensions of the three tubes are given
and were measured after fabrication.

| | | | | | curved | |
|---|---|---|---|---|---|---|
| | ID | OD | | | section | radius |
| Tube | (mm) | (mm) | straight section (mm) | | (mm) | (mm) |
| Inner | 0.80 | 0.92 | 192 + 137 | (passive) | 65 | 52 |
| Middle | 0.97 | 1.10 | 113 + 23 | (passive) | 70 | 87 |
| Outer | 1.20 | 1.40 | 100 + 6 | (passive) | — | — |

Additional passive parts were added to the straight sections for easier accessibility to the tube from the proximal end of the robot and for secure placement within the tube grippers. In the example prototype, these passive parts did not experience any force or torque from interaction with the environment or other tubes.

The outer tube can be firmly attached to the distal end of the robot, for example, using a clamping shaft collar 122. The distal end 106 of the ACR 100 can be linked to the body. In one, non-limiting example, the link can be formed through an ATI MINI40 6-DOF force/torque sensor 124 (ATI Industrial Automation, NC). This configuration can be used to monitor the insertion forces on the outermost tube during the ACR deployment.

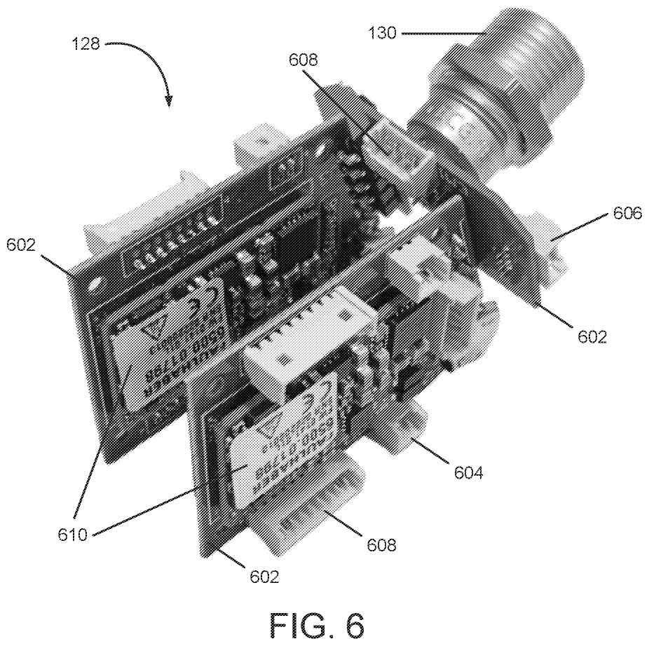

An alternative design of the ACR 103 is shown in FIG. 1B. Descriptions of the components with the same reference numbers in FIG. 1A are identical structures are not repeated here. In a non-limiting example, the ACR 103 includes a custom printed circuit board (PCB) 128 at the proximal end of the housing, proximal to the two rotation motors 108 and two translation motors 110. The PCB 128 will be described in further detail with reference to FIG. 6. The PCB 128 accommodates at least four miniature motion controllers, which are external components for the configuration of ACR 100. It facilitates connections for the motors, encoders, reference and CTR tip electromagnetic tracking sensors, alongside buttons for a user interface, and allows for a computer connection via a single external connector 130 and cable 131 at the robot's end, extending proximally from the housing 102. This configuration employs a 2-wire CAN bus connection for commanding and reading all motion controls, significantly reducing the number of conductors needed for the cable. Now, the cable consists of 13 conductors, including three grounds, +6V and +24V power lines, seven signal lines for EM tracking sensors, two lines for the CAN bus, and a single line for emergency stop button. The cover for this section is tapered and bent for better ergonomics. The PCB 128 connects to an external connector 130 extending distally outside of the housing 102 to connect to external processing components and a power supply.

In a non-limiting example, the ACR 103 further includes a proximal supporting plate 132 between the motors 108, 110 and the carriage units 116, 118 to accommodate proximal ball bearings 134. At the distal end of the carriage units 116, 118, a distal supporting plate 136 accommodates distal ball bearings 138 and collar clamps 140. The supporting plates 132, 136, ball bearings 134, 138, and collar clamps 140 eliminate axial and radial play during actuation of the carriage units 116, 118.

In a non-limiting example, ACR 103 includes a tapered 3D-printed cap 142 at the distal end 106 of the housing (no force sensor 124 as in FIG. 1A). The tapered cap 142 reduces weight and interference with the electromagnetic field used for tracking to enhance the positioning tracking accuracy of the ACR 103 and tip and the distal end of first cannula tube 112. In a non-limiting example, reference EM sensor 126 is positioned within the tapered cap 142, allowing for a reduction in the tracking interference from the carriage units 116, 118, which contain metallic gears and bearings.

In a non-limiting example, ACR 103 includes six landmark grooves 144 integrated within the housing 102 for landmark registration of the ACR frame in the reference sensor's frame, as will be described in further detail.

FIGS. 1C-1F provide another ACR 105 design, wherein the housing 102 is reversibly separable into a proximal portion 146 and a distal portion 148. Descriptions of the components with the same reference numbers in FIGS. 1A-1B are identical structures are not repeated here. This design allows the proximal portion 146 to be sterilized, while the non-sterilizable distal portion 148 can be enclosed in a sterilized bag or a hard shell to isolate the non-sterile environments. In a non-limiting example, the two portions 146, 148 are easily connected by a push, secured by two locking mechanisms that ensure a tight junction (not shown). Furthermore, the locking mechanism is disengaged by simultaneously pressing two lock buttons on the distal portion 148, allowing for quick and safe separation of the sections.

In a non-limiting example, the proximal portion 146 includes the pair of rotation motors 108 and the pair translation motors 110. It may further include the PCB 128 and external connector 130 as described above for FIG. 1B. The proximal portion 146 further includes a first coupling 149, 150, 152, 154 operably connected and distally positioned to each motor of the pair of rotation motors 108 and the pair of translation motors 110. In a non-limiting example, the first coupling 149 is connected to the rotation motor 108 for the first carriage unit 116. The first coupling 150 is connected to the translation motor 110 for the first carriage unit 116. The first coupling 152 is connected to the rotation motor 108 for the second carriage unit 118. The first coupling 154 is connected the translation motor 110 for the second carriage unit 118. Each of the first couplings 149, 150, 152, 154 includes one or more spring-loaded pins 156 configured to engage corresponding sockets (not shown) in second couplings 158, 160 connected to and proximally positioned to each carriage unit 116, 118. The second couplings 158, 160 are connected the first carriage unit 116. Two additional couplings (not shown in FIG. 1C) are connected to the second carriage unit 118. The connection between each first coupling and each second coupling provides a mechanical connection for the pair of rotation motors 108 and the pair of translation motors 110 to actuate the carriage units 116, 118. Further, electronic connectivity for the EM sensor(s) is achieved through the connection between the first couplings and second couplings.

In a non-limiting example, the proximal portion includes one or more control knobs for manual control of the ACR 100. The one or more control knobs may include a joystick or buttons. In one embodiment, a first control pad 162 may comprise five buttons for planar navigation and confirmation are located on the surface of the proximal portion. When held, the thumb of an operator covers and presses the buttons of the first control pad 162. The second control pad 164 may include two buttons for insertion and retraction and is located on the surface of the proximal portion 146, opposite the first control pad 162. When held, the index finger of an operator covers the and presses the buttons of the second control pad 164. The first and second control pad 162, 164 allows an operator to manually navigate and adjust the ACR target trajectory during procedures.

In a non-limiting example, the distal portion 148 includes the first carriage unit 116 and second carriage unit 118. In a non-limiting example, the distal portion 148 may include the distal end 106 of the ACR 103 as shown in FIG. 1C. Alternatively, the distal portion 148 may include the distal end 106 of the ACR 100.

In a non-limiting example, the distal portion 148 includes a channel 166 coupled to the outer surface. An active cannula tube comprising first cannula tube 112 and second cannula tube 114 may be introduced into the distal portion 148 by the channel 166 and extend longitudinally therein and distally outside the distal end 106. The channel 166 provides easy access to the proximal end of the distal portion 148, without risking exposure to the non-sterile environment of the proximal portion 146. This channel bends and guides the first and second cannula tubes 112, 114 through the tube actuation section of first and second carriage units 116, 118.

The ACRs of FIGS. 1A-IF are non-limiting, and the structures may be interchangeable between ACR 100, 103, and 105. In non-limiting example, the ACR 100 of FIG. 1A may be include a PCB 128 and/or be separable as ACR 105. Likewise, ACR 103 and 105 may include distal ends 106 with force sensors 124 as in FIG. 1A. In another example, ACRs 100 and 103 may include channel 166 for feeding the active cannula tubes 112, 114 into the distal ends 106.

In another non-limiting example, the ACR 100, 103, 105 may include a foot pedal (not shown) acting as a clutch, which must be pressed while pressing control pads 162 and/or 164 to actuate the cannula tubes. For example, the second control pad 164 can work in conjunction with the foot pedal for additional safety and to prevent accidental insertion or retraction of the ACR during manual operation.

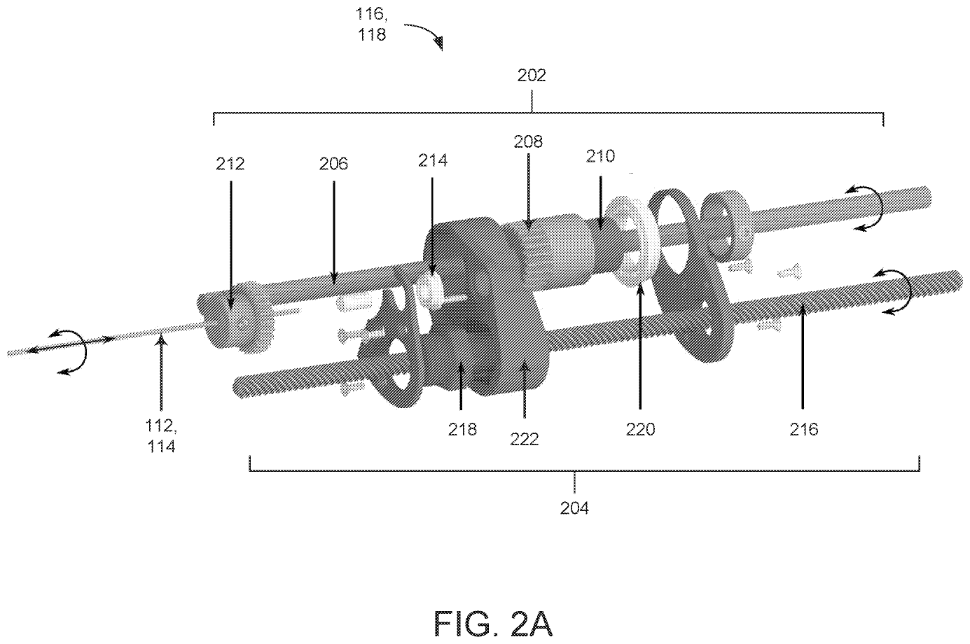

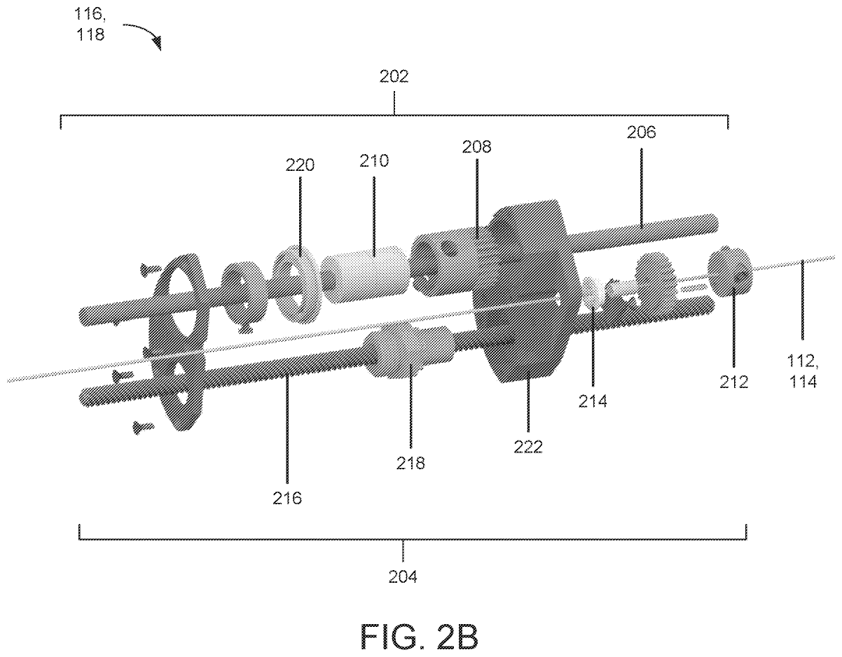

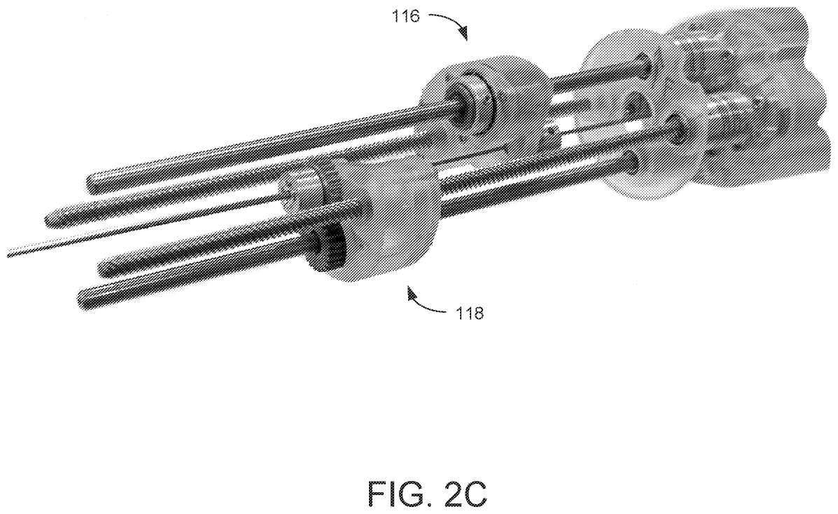

Referring to FIGS. 2A-2C, a non-limiting example of the cannula tube 112, 114 and carriage unit 116, 118 is shown. The carriage unit 116, 118 includes a rotation unit 202 and translation unit 204. A variety of configurations can be used to implement the systems and methods described herein. Thus, the particular configurations illustrated and described with respect to FIGS. 2A-2C is just one example of a variety of configurations or implementations that can be created based on the systems and methods described herein.

In a non-limiting example, the rotation unit 202 includes a splined shaft 206. A first gear 208 is positioned concentrically around the splined shaft 206. A linear bearing 210 is also positioned concentrically around the splined shaft 206, and within the first gear 208. A bearing 220 is concentrically positioned around the first gear 208. A shaft collar 212 is concentrically positioned around the cannula tube 112, 114 and includes teeth that are configured to engage the teeth of the first gear 208. A bearing 214, for example a tube bearing, may be concentrically placed around the cannula tube 112, 114. Rotation of the splined shaft 206 by a rotation motor 108 causes the rotation of the first gear 208, the shaft collar 212, and the cannula tube 112, 114.

In a non-limiting example, the translation unit 204 includes a lead screw 216, a flanged nut 218 positioned concentrically around the lead screw 216.

A linker 222 links the rotation unit 202 and translation unit 204. The linker 222 contains two openings through which both the splined shaft 206 and lead screw 216 extend.

During translation, a translation motor 110 rotates the lead screw 216, which causes a translational displacement of the flanged nut 218. The translation is guided by splined shaft 206 and linear bearing 210.

The motion of both cannula tubes 112,114 is delivered by their respective carriages 116, 118. In a non-limiting example, the translation of the carriages is guided by a 4 mm diameter splined shaft 206 and a linear bearing 210. The translational motion is achieved through a flanged nut 218 paired with a 4 mm diameter fast-travel ultra-precision lead screw 216 with 10 mm travel per turn. Both the linear bearing 210 and the flanged nut 218 are secured to the respective carriages as seen in FIGS. 2A-2C. In a non-limiting example, the rotation of each tube is powered by a pair of 24-tooth gears, with one gear attached to the tube via a shaft collar 212 and the other attached to the splined shaft 206, with a linear bearing 210 in between to reduce friction.

The splined shaft 206 provides not only linear guidance for the carriage but also transfers rotational motion to the carriage. In a non-limiting example, the two splined shafts 206 and lead screws for each of the cannula tubes 112, 114 are secured at the distal end 106 with ball bearings (not shown) and connected to an individual motor through a shaft coupling at the proximal end.

The actuation of cannula tubes 112 and 114 as described in the non-limiting example above, in addition to the lateral and rotational motion of the ACR 100 itself, may result in 6 DoF motion of the distal tip. In this example configuration, each cannula tube 112, 114 may be actuated in 2 DoF, namely translation along the length of the housing and rotation about the axis running the length of the housing 102 by the carriage units 116 and 118, respectively. In this non-limiting example, cannula tubes 112 and 114 have curved distal sections which allows for various bending configurations and positions of the distal tip up to 4 DoF due to the interaction between the flexible cannula tubes 112 and 114. The lateral positioning of the of the entire ACR 100 device by the user accounts for 5-DOF and the rotation of the ACR accounts for 6-DOF during the deployment phase in this non-limiting example.

To reduce weight and ensure that extended use is non-taxing, even when used via a single hand over an extended medical procedure, a variety of configurations can be used. In one example, four CXR 1727 DC micromotors (Faulhaber, Baden-Wuerttemberg, Germany) were used to actuate the lead screws 216 and the splined shafts 206, each paired with a corresponding 91:1 planetary 17/1 gearhead (Faulhaber, Baden-Wuerttemberg, Germany) and an IEH3-4096L encoder (Faulhaber, Baden-Wuerttemberg, Germany). This example configuration reduces the volume of the ACR over larger motor or control systems.

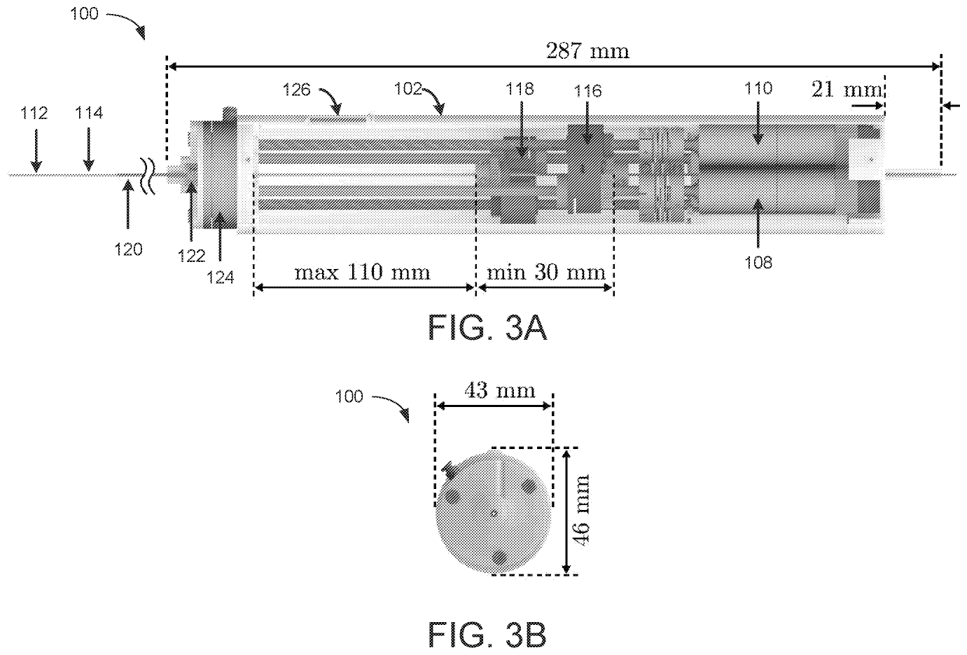

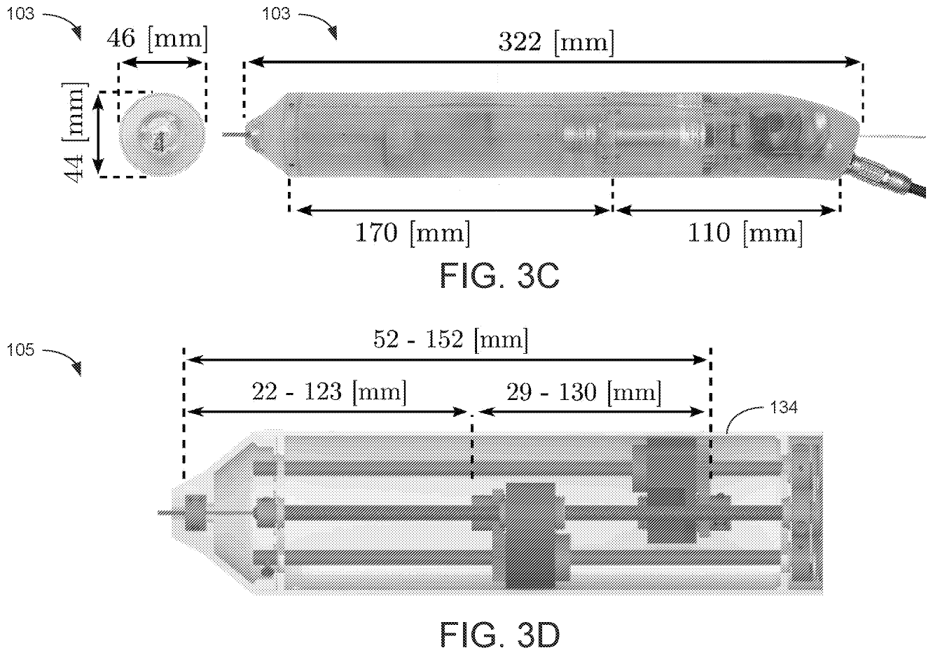

As described above, a prototype was created that illustrates the ability to utilize the systems and methods provided herein to create a device that can be held and controlled by a clinician using a single hand over an extended medical procedure. To that end, FIGS. 3A-3B shows a non-limiting example of the dimensions of the example, hand-held ACR 100. The length of the housing 102, excluding the tubes, is 287 mm, as illustrated in FIG. 3A. This design allows for 110 mm of linear motion and unlimited rotation for the inner and middle cannula tubes 112, 114. However, the distance between the base of the inner tube (first tube 112) and middle tube (second tube 114) active section is limited to 30 mm due to the physical collision of the carriages. The housing 102 of the robot has an elliptical cross-section with a minimum diameter of 43 mm and a maximum diameter of 46 mm as illustrated in FIG. 3B.

FIGS. 3C and 3D illustrate the non-limiting dimensions of ACR 103, 105. An additional section measuring 80 mm is added to the proximal end to accommodate the PCB.

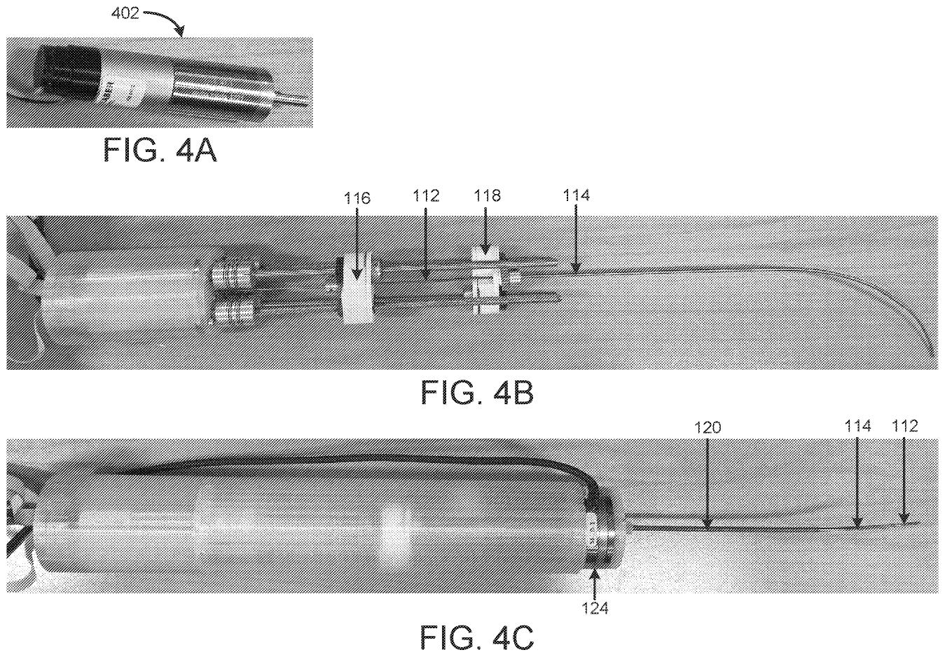

Photographs of components of the non-limiting example prototype are shown in FIGS. 4A-4C. The housing of the ACR is made of two cylindrical pieces. One covers the actuation section and houses the shaft bearings, and the other covers and holds all four motors 402. A longitudinal canal is designed in the center of the motors section to guide the inner cannula tube 112 out of the proximal end 104 of the ACR. This facilitates the insertion of the fiber optic and electromagnetic (EM) sensors through the inner tube from the proximal end of the robot through the procedure (not shown). All designed parts including the housing 102 of the ACR 100, front and end caps and the body of the carriage units 116, 118 may be fabricated using stereolithography 3D printing.

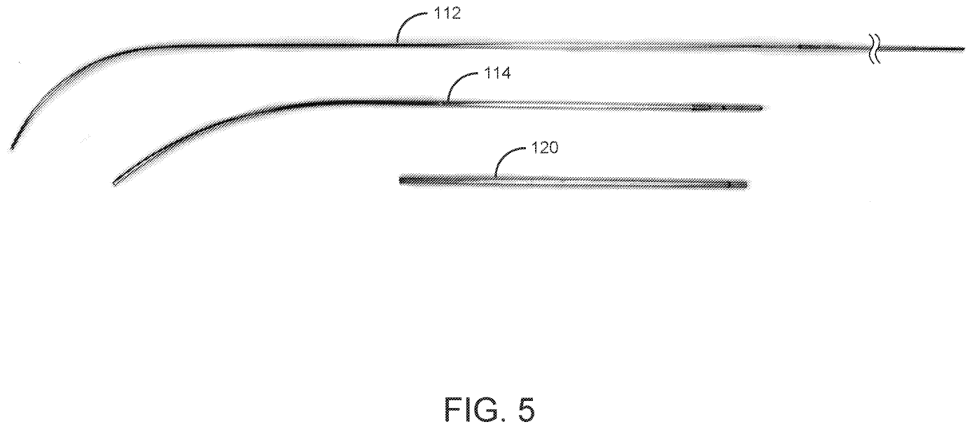

FIG. 5 shows the first cannula tube 112, second cannula tube 114, and third cannula tube 120. The cannula tubes 112, 114, 120 may be made of or include a flexible material. For example, the flexible material may include a memory material. In one example, the memory material may include Nitinol. In a non-limiting example, the material stiffness enables the puncturing of dermal or renal tissue. In a non-limiting example, the first and second cannula tubes 112, 114 are fabricated from straight Nitinol tubes bent using heat treatment. The first and second cannula tubes 112, 114 include straight sections in their proximal ends and curved sections in their distal ends external to the housing 102. In this particular configuration, the third cannula tube 120 forms a straight section.

FIG. 6 shows a detailed view of PCB 128. The PCB includes at least three custom distributions 4-layer PCBs 602 including a motor connector 604 for operation of rotation motor(s) 108 and translation motor(s) 110. The 4-layer PCBs 602 further include an ACR tip EM sensor connector 606 and reference EM sensor port 608 configure to operably connect to the ACR distal tip EM sensor and reference EM sensor (i.e., reference EM sensor 126). The PCB 128 further includes at least four miniature motion controllers (MC3001B) 610. External connector 130 connects the PCB 128 to processor and power supply.

The PCB 128 facilitates connections for the motors, encoders, reference and CTR tip electromagnetic tracking sensors, alongside buttons for the user interface, and allows for a computer connection via a single connector at the robot's end. This configuration employs a 2-wire CAN bus connection for commanding and reading all motion controls, significantly reducing the number of conductors needed for the cable in an ACR without the PCB 128. In ACRs of FIGS. 1B and 1C, the cable 131 consists of 13 conductors, including three grounds, +6V and +24V power lines, seven signal lines for EM tracking sensors, two lines for the CAN bus, and a single line for emergency stop button.

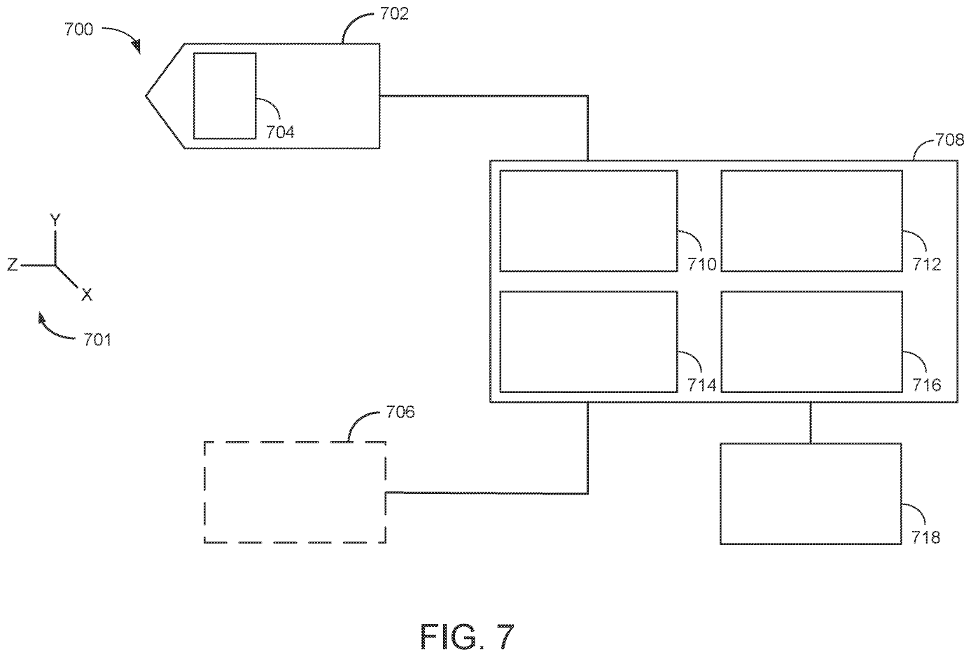

Referring to FIG. 7, a non-limiting example of a the ACR system 700 is described. The ACR system 700 includes an ACR 702, such as ACRs of FIGS. 1A-1F. In a non-limiting example, the ACR 702 includes one or more EM sensors 704. The EM sensor may include a reference EM sensor 126, or a distal EM sensor 125 embedded in the distal tip of first cannula tube 112. The ACR system 700 further includes an optional EM tracker 706 configured to localize the ACR and/or first cannula tip with a frame of reference 701.

The ACR 702 is operably coupled to a processor 708, which houses a plurality of control units. In a non-limiting example, the processor 708 may include a force sensing unit 710, operably coupled to a force sensor such as force sensor 124 in FIG. 1A. Further, processor 708 may include a motor controller 712 operably coupled to rotation and translation motors, such as rotation motors 108 and translation motors 110 in FIGS. 1A-1C. In a non-limiting example, processor 708 also includes a navigation system 714, whose functions are described in detail in the example below. Additional control units 716 may be included in the processor 708 to provided additional operation of the ACR. In a non-limiting example, a user interface 718 is operably coupled to the processor 708. The user interface may be integrated into or separate from the processor 708. In a non-limiting example, the user interface 718 includes a computer, display, keyboard, touchpad, buttons, foot pedal, joystick or other device for receiving user input and/or displaying tracking information, ACR operation data, or other relevant information for a PNCL procedure.

In a non-limiting example, the ACR system 700 includes a USB-CAN 722 connected to the ACR 702, for commanding and reading all motion controls between the processor 708 and the ACR 702.

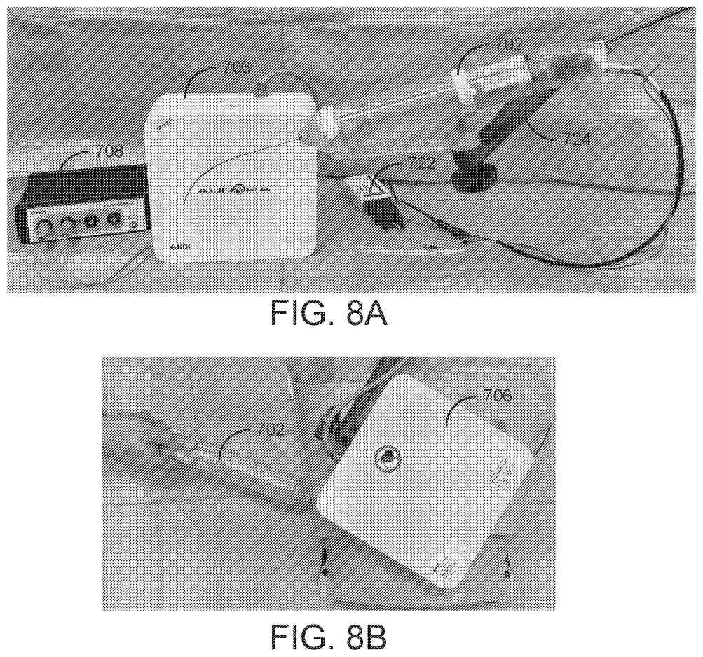

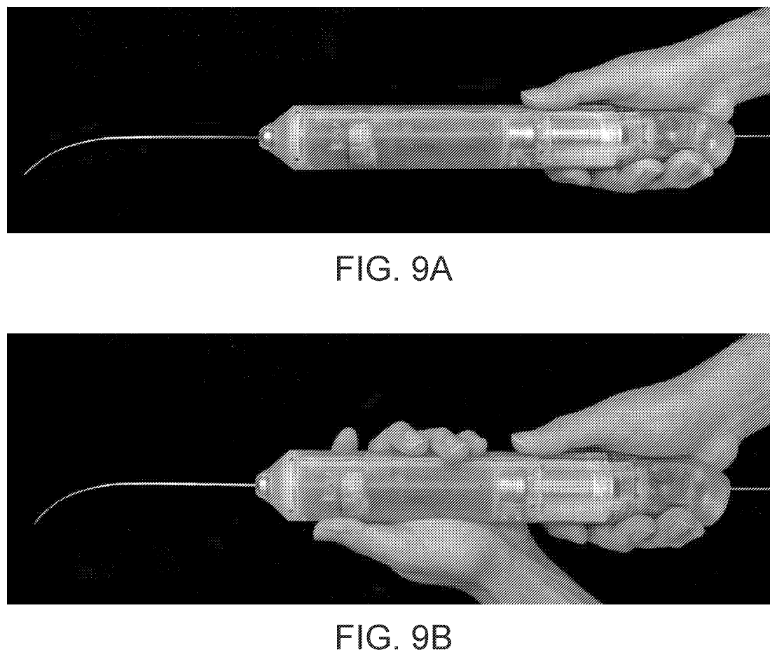

FIG. 8A provides a non-limiting example of the ACR system 700 using a passive arm 724, while FIG. 8B shows the ACR system 700 being manually operated.

FIGS. 9A-9B provide an example of a single handed and dual handed grasp of a ACR according to embodiments herein.

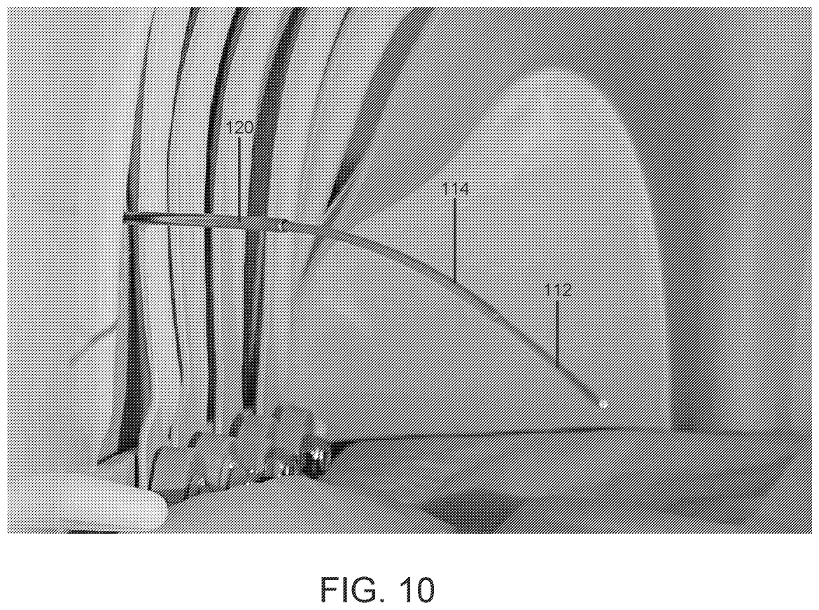

FIG. 10 shows an example configuration of the the extended first cannula tube 112, second cannula tube 114, and third cannula tube 120 inside a phantom.

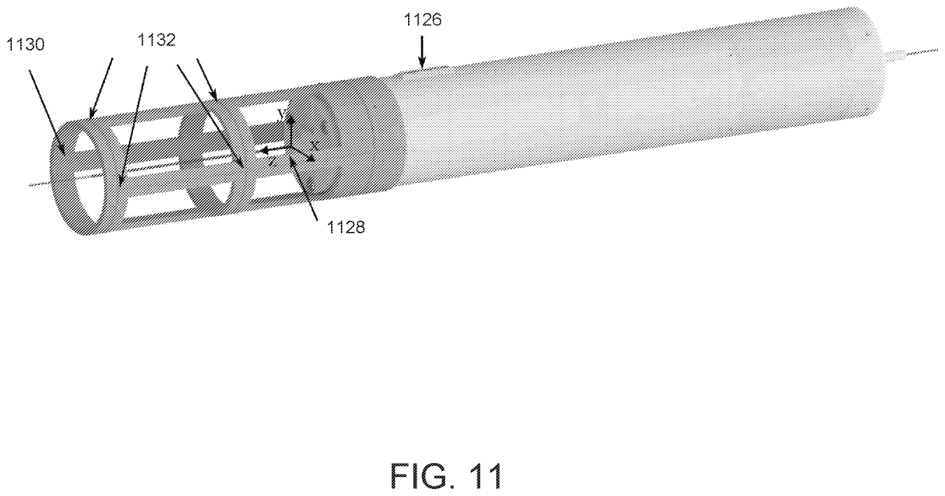

In a non-limiting example, the position of the ACR tip can be monitored using electromagnetic sensors. A 5-DoF sensor may be installed inside the distal end of the inner tube (not shown), and a 6-DoF reference sensor 526 may be installed on the housing of the robot (e.g., refence EM sensor 126 in FIG. 1). The exact transformation matrix of the ACR coordinate frame 1128 in the reference EM sensor may be calculated through an 8-point registration process using a registration jig 530 as shown in FIG. 11. The location of a predesigned landmarks 1132 may be recorded using a 5-DoF sensor along with the location of the reference sensor 1126.

Alternatively, the landmark grooves 144 depicted on ACR 103, 105 may be recorded using a 5-DoF sensor along with the location of the reference sensor 1126. Since the transformations from the coordinate frame to the landmarks may be known from the CAD files and the transformations of the reference sensor and the landmark sensor are measured using EM tracking in the EM coordinate frame, the transformation from the reference sensor to the ACR's coordinate frame is calculated. Alternatively, the transformation between the reference sensor and the ACR coordinate frame could also be calculated from the CAD model.

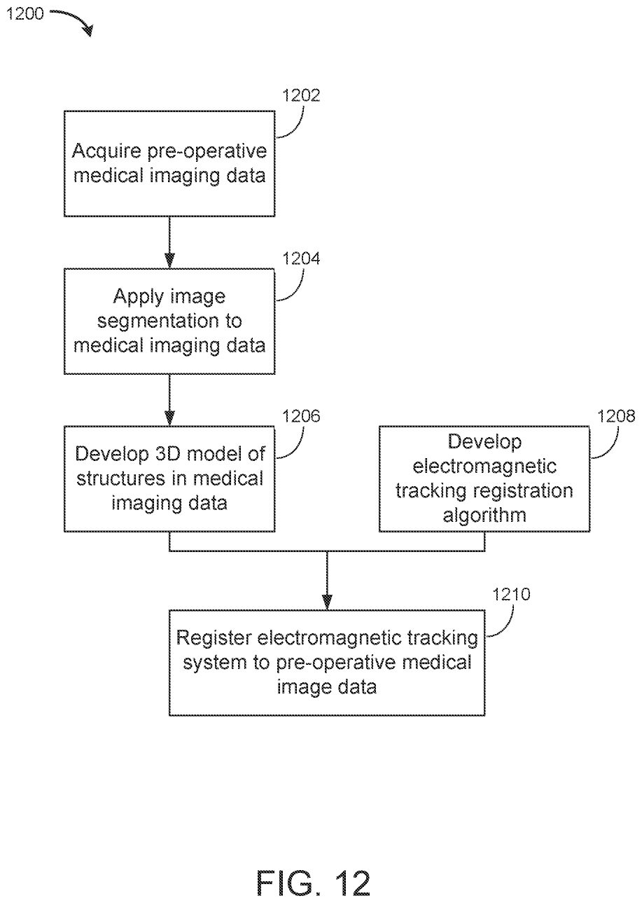

FIG. 12 shows a non-limiting method 1200 of the development and operation of a navigation system in the ACR system. At step 1202, pre-operative medical imaging data is acquired. The medical image data includes, but is not limited to, MRI data or CT imaging data. At step 1204, the medical imaging data from step 1202 is segmented to identify structures in the medical imaging data. Next, at step 1206, the segmented medical imaging data is used to develop a three-dimensional (3D) model of the structure. At step 1208, an electromagnetic tracking registration algorithm is developed. The algorithm may be developed based on the determination of the ACR coordinate frame 1128 described in FIG. 11. Step 1208 may occur concurrently with the execution of steps 1202-1206. Alternatively, step 1208 may be performed before or after the execution of steps 1202-1206. At step 1210, registration of the 3D model from step 1206 and the registration algorithm of step 1208 is performed and used to provide precise guidance the ACR under real-time imaging or context-specific diagnostic imaging registered to the coordinates of the ACR device. This method 1200 is described in further detail below.

Non-limiting examples of ACR system control strategies are described in the example below.

### Example 1

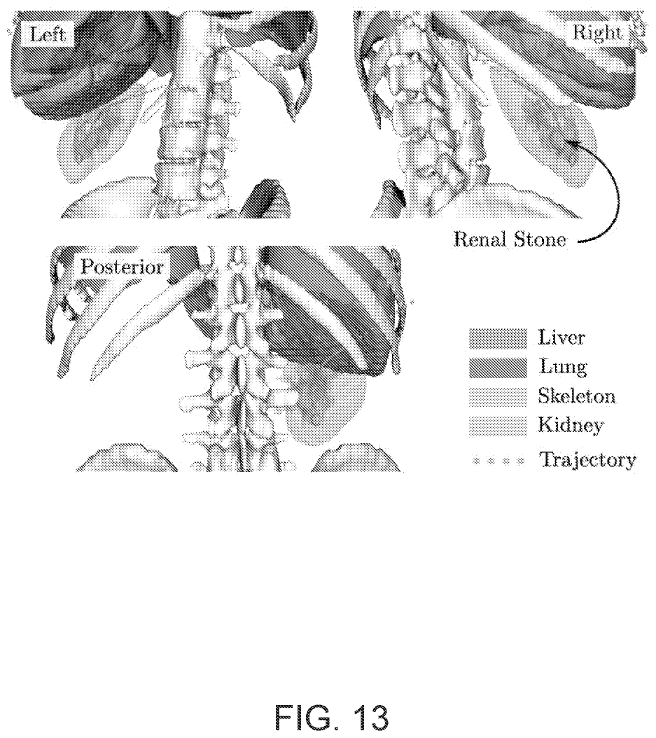

A compact, hand-held four-degrees of-freedom (DoF) CTR specifically designed for PCNL procedures is described herein. The design prioritizes ease of use in the operating room and surgeon comfort, making it one of the most compact CTRs currently available, capable of penetrating up to 100 mm from its retracted position. The CTR was designed to be close to conventional PCNL devices to streamline autonomous access to renal stones from the skin surface. The aim was to leverage a preoperative trajectory generated through an image-guided surgical planning algorithm as shown in FIG. 13.

### System Design

Given the intended application of the proposed CTR as a hand-held steerable device to be used in PCNL procedures, the primary design considerations include ensuring compact dimensions, minimized weight, and improved ergonomics. These aspects are crucial for seamless integration into the operating room setting and to minimize the surgeon's hand fatigue. In order to achieve optimal ergonomics, a slightly oval cylindrical design was adopted to facilitate a power grip. Additionally, any external connection to the robot was kept to a minimum to enhance the aforementioned design requirements. In order to enable access from the skin surface to the collecting system of the kidney and, consequently, the renal stones, the robot is required to translate and rotate two pre-curved tubes for the necessary flexibility to precisely follow the required trajectory. Additionally, the proximal end of the inner tube must be accessible for the insertion of an electromagnetic (EM) tracking sensor or a laser fiber for ablating and treating renal stones.

### Mechanical Design

A four-DoF robot was developed to accommodate three tubes (FIG. 1B), with the outer tube remaining stationary while both the middle and inner tubes undergo translation and rotation. In order to attain the required elliptic cylindrical design with a minimized diameter, a parallel actuation system design approach was used (FIG. 2B). This design allows for the positioning of all four motors and gearboxes, the heaviest components of the robot, at the proximal part directly beneath the surgeon's palm. This positioning aims to mitigate hand fatigue during operation by minimizing the torque on the surgeon's wrist due to weight. Additionally, separating the motor section and localizing it at the proximal end of the robot helps reduce interference from the EM tracking system, which is elaborated upon herein.

The tube actuation mechanism of the robot consists of two carriage modules, each designed to independently rotate and translate the corresponding tube: one for the inner tube active section and another for the middle tube (see FIG. 1B). As shown in FIG. 2B, each carriage comprises three main components: a tube holder collet, a lead nut, and a linear bearing. The linear bearing is mounted on a 4 mm splined shaft, while the lead nut is affixed to a 4 mm diameter lead screw with a 10 mm pitch, facilitating fast travel. This assembly converts the rotational motion of the actuator into linear motion and translates the carriage along the associated tube. The linear bearing serves a dual purpose: it provides linear guidance for the carriage and transmits the motor's rotational motion to the tube. As illustrated in FIG. 2B, the linear bearing is connected to the carriage via a rotational bearing allowing the linear bearing to rotate in sync with the splined shaft. The tube holder collet, attached to the carriage with a rotational bearing, engages in rotational movement with the linear bearing through a spur gear mechanism. The outer tube is securely attached to the distal end of the robot using a screw-tightened collet. FIG. 2C shows the fabricated tube actuation mechanism.

The entire concentric tube manipulation module is enclosed within a 1.4 mm thick cylindrical shell with an oval cross-section, featuring diameters of 44 mm and 46 mm, as shown in FIG. 3C. This specific cross-section was optimized using the cost function of the area of the cross-section, and the constraints of enclosing all actuators with a minimum 1.4 mm thick shell and keeping an oval cross-section (i.e., maintaining two lines of symmetry: the vertical line through the center, and the horizontal line through the center) for ergonomic considerations. Both the splined shafts and the lead screws are mounted on the shell using rotational bearings. To prevent any axial play, which could compromise the linear positioning accuracy, the lead screws are axially supported at both ends. This support is crucial as the translation force resulting from the tube interactions could otherwise impact accuracy. FIG. 3D shows the actuation range of the design. The splined shafts and the lead screws are connected to the motors using flexible couplings. The structural and mechanical parts excluding the standard mechanical parts (bearings, couplings, lead screws, and gears) were fabricated using stereolithography 3D printing.

All four-DoFs are actuated using four CXR 1727U024 motors (Faulhaber, Baden-Wuerttemberg, Germany), each coupled with a 91:1 planetary 17/1 gearhead and an IEH3-4096L incremental encoder, also from Faulhaber. The motors and gearboxes are compactly housed within the shell. The proposed design allows for maximum velocity of insertion and rotation of the tubes equal to 10 mm/s and 3.4 rad/s, respectively.

A longitudinal pathway was designed along the robot guiding the proximal end of the inner tube towards the back of the robot. This design ensures an unobstructed path for the inner tube to provide access for inserting a laser fiber or EM tracking sensor into the tube.

### Nitinol Tube Bending

Nitinol tubes were chosen over plastic tubes because of their higher stiffness, which is essential for both skin and renal puncture. Straight Nitinol tubes were pre-curved using heat treatment. The traditional methods such as furnace heating and Joule heating, commonly mentioned in the literature, were unsuitable for this design. The length of the inner tube, at 500 mm, would require an oversized air furnace for uniform heating, which was impractical. Additionally, the axial thermal expansion of the tubes during the Joule process caused distortions near the clamps, affecting the tube's integrity, especially for the inner tube which has a smaller diameter.

To ensure a practical, and cost-effective approach for tube bending, a heat gun was employed for locally heating the curved sections of the tubes. Bending molds with groove properties tailored to the tube diameter and required curvature were fabricated using natural air drying Activ-Clay (Activa, TX, USA). The tubes were heated by the heat gun at 480 degrees Celsius over the curved section for at least 30 minutes. The tubes were then left to cool in the mold through natural convection. FIG. 5 shows the fabricated tubes. The parameters of the set of tubes are shown in Table II. The aforementioned method led to a curvature error less than or equal to 5%. This error is attributed to the clay mold's shrinkage during the drying process and the tube's springback after heat treatment.

TABLE II
Tube Set Parameters

| Parameter | inner | middle | outer |
|---|---|---|---|
| Inner Diameter [mm] | 0.74 | 0.97 | 1.45 |
| Outer Diameter [mm] | 0.94 | 1.37 | 2.05 |
| Active Straight Length [mm] | 151 | 61 | 62 |
| Curved Length [mm] | 58 | 78 | 0 |
| Intended Curve Radius [mm] | 40 | 100 | — |
| Measured Curve Radius [mm] | 41 | 95 | — |
| Passive Straight Length [mm] | 290 | 16 | 6 |

### Registration Landmarks

In order to detect the transformation of the robot frame in space, and to track the transformation of the CTR tip with respect to the robot frame, two Aurora EM tracking sensors (NDI, ON, Canada) were employed. A 6-DoF 610094 sensor was placed at the distal end of the robot shell, serving as a reference sensor to localize the robot frame (see reference EM sensor 126 of FIG. 1B). The robot frame was registered in the reference sensor frame using the landmark registration method with 6 landmarks built into the body of the robot (see landmark grooves 144 of FIG. 1B). A 5-DoF 610158 sensor was inserted into the CTR's distal end from the proximal end of the inner tube, and the inner tube was connected to a sensor shield to mitigate interference from the motors. Positioning the motors and electronics at the proximal end of the robot and the reference sensor at the distal end allows the insertion of the distal half of the robot into the EM field while keeping the motors and electronics out of the EM tracking volume. This ensures precise localization of both the robot and the CTR's end-effector.

### Electronics

The lower-level motor control is performed by four MC3001B motion controllers (Faulhaber, Baden-Wuerttemberg, Germany) integrated at the back of the robot. A custom-designed main PCB board was developed to simplify the connections between the motors and encoders with the motion controllers, as shown in FIG. 6. Integrating these motion controllers into the robot significantly reduced the complexity of the cabling. The number of required cable conductors for the actuator section decreased from 34 (8 for power and 26 for encoder signals) to 5 (2 for power, 2 for the CAN bus, and 1 for the emergency stop button), thereby enhancing the maneuverability of the robot in the OR. The connections for the power line, CAN bus, emergency stops, and two electromagnetic tracking sensors were provided through a single 16-pin LEMO connector at the back of the robot.

Communication between the motion control system and the high-level controller running on a desktop computer was established using IXXAT USB-to-CAN (HMS, Halmstad, Sweden) through the CANopen communication protocol with synchronized CiA301 application layer.

### Ergonomic Analysis

Research has indicated that ergonomic considerations for surgical instruments have been underemphasized, contributing to widespread work-related musculoskeletal complaints among surgeons. This is particularly the case for MIS, where the physical strains imposed on surgeons' bodies are significantly higher. Robot-assisted surgery has offered promising improvement in this area by providing better posture, leading to less postoperative discomfort and muscle strain. The ergonomic significance is further heightened in the context of a hand-held robotic system since the surgeon physically supports the entire apparatus.

While a precision grip involving the fingers offers superior positioning accuracy compared to a power grip, there are design factors, such as the space needed for the mechanical mechanism and electronics that limit miniaturization of a hand-held robot to be utilized with a precision grip, thereby pushing the design towards a power grip approach. The Canadian Centre for Occupational Health and Safety recommends that a tool intended for one-handed operation must not exceed 1.4 kg in weight. Moreover, they recommend a circular or oval cross-section with a diameter of 30 to 50 mm and a length of more than 100 mm for an effective power grip. In it has been shown that surgeons' preferences for handle size, ranging from 30 to 40 mm in diameter, correlate with their hand size, categorized from extra small to large. In line with these ergonomic considerations, the robot design weighs 570 g and incorporates an oval cross-section with diameters of 44 mm and 46 mm, closely aligning with the preferred range for hand-held surgical instruments.

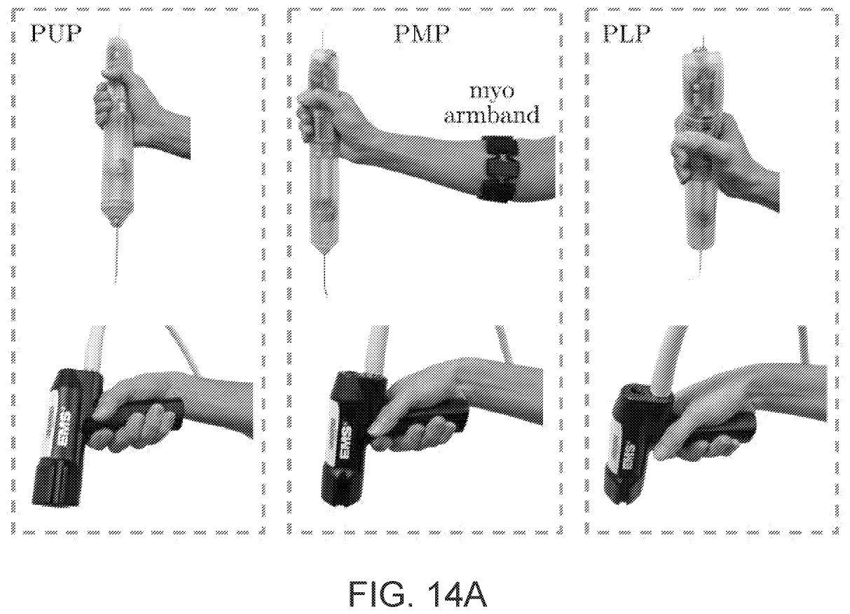

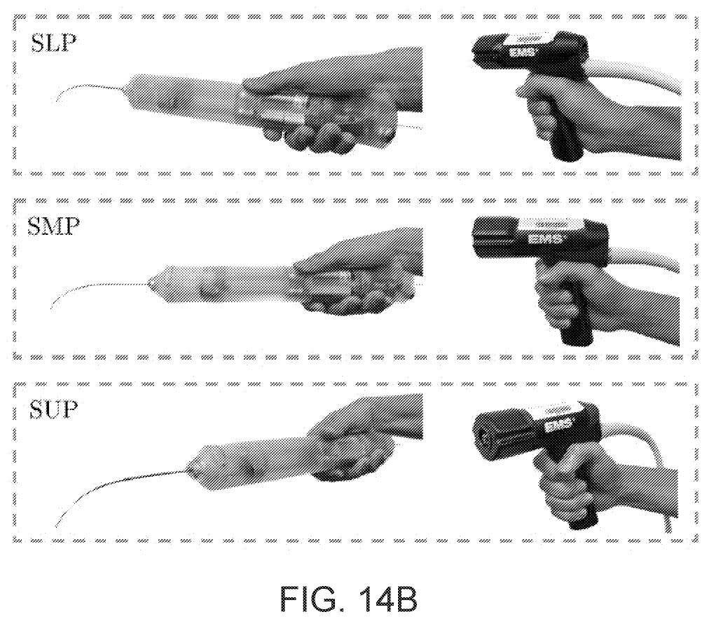

The level of muscle activation while handling a device could serve as a representation of its ergonomics. Surface electromyography (sEMG) has been used to optimize the ergonomic design through minimizing muscle activation. In order to further evaluate the ergonomics of the proposed design, the muscle activation experienced by users was compared when handling the designed PCNL robot against that induced by a LithoClast handpiece (Electro Medical Systems, Switzerland), a 660 g hand-held lithotripter device which is routinely used for ablating renal stones in PCNL procedures. The users were asked to hold both the LithoClast and the hand-held CTR in orientations replicating those typically assumed in the OR, including prone lower pole (PLP), prone mid pole (PMP), prone upper pole (PUP), supine lower pole (SLP), supine mid pole (SMP), and supine upper pole (SUP) positions, as shown in FIGS. 14A-14B. A group of 6 urologists and 5 non-clinicians participated in the experiments. Muscle activation was recorded using a MYO armband (Thalmic Labs, ON, Canada) placed on the forearm, with an average sampling frequency of 330 Hz. The sequence of device utilization (robot or LithoClast) was randomized to alleviate the influence of fatigue. It is important to highlight that one of the surgeons preferred to grasp the body of the LithoClast, rather than the handle, during all prone positions due to superior ergonomics.

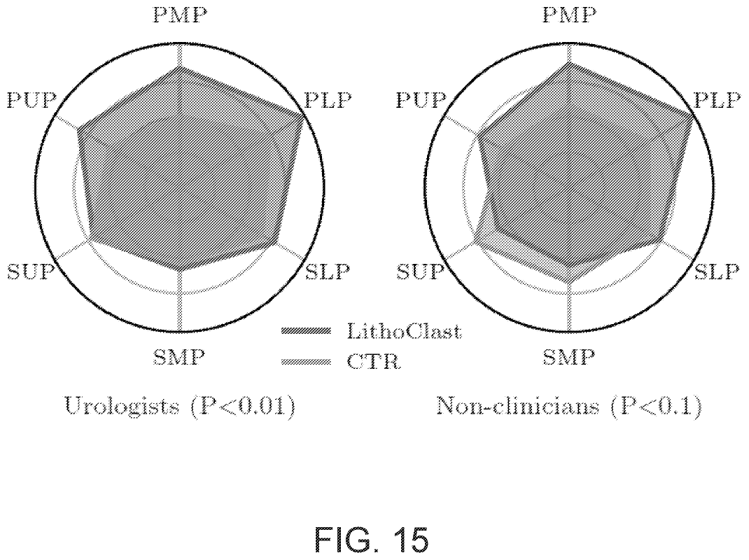

FIG. 15 shows the normalized mean absolute value of the eight signal channels recorded by the MYO armband sEMG module. The analysis indicates that, among the urologists, the average muscle activation is either equal to or lower when using the CTR compared to the traditional LithoClast handpiece. Similarly, among the non-clinicians, the average muscle activation is lower when using the CTR compared to the traditional LithoClast handpiece across various orientations, except for the SUP and SMP.

Statistical t-tests, demonstrate strong evidence of significantly less muscle activation among urologists (p<0.01) and weak evidence of less muscle activation among non-clinicians (p<0.1) when using the CTR. This difference between urologists and non-clinicians might be because of the more optimized way of grasping devices used by urologists in comparison to non-clinicians. These results suggest that the proposed robotic tool does not increase the strain level on the muscle compared to the conventional handpiece devices used in PCNL procedures.

The primary difference between the LithoClast group and the CTR group is because of the lighter weight of the robot (570 g) in comparison to the LithoClast (660 g plus a heavier cable) coupled with the superior mass distribution of the CTR. This design localizes the center of mass beneath the user's palm, thereby reducing excessive torque on the wrist. The reduced muscle activation observed with the LithoClast in the supine orientation is due to the pistol-like shape, which makes it more suitable for supine orientation, ensuring a neutral wrist posture. However, this configuration results in a flexed wrist when the patient is in a prone orientation (one of the surgeons avoided this unfavorable wrist posture by grasping the body of the LithoClast in all prone orientations). In contrast, the slightly oval cross-section of the design of the CTR leads to a more favorable wrist posture in the prone orientation, but less favorable in the supine orientation.

### Kinematic Model of CTRs

Each tube has two degrees of freedom resulting, in the most general case, in a total of 2n independent inputs for an n-tube CTR. The joint space vector was defined as the n-dimensional vector that represents the translation and rotation values for each tube within the robot assembly, denoted as q¹=[δ₁, . . . , βₙ, α₁, . . . , αₙ], where βᵢ and αᵢ denote the linear and angular actuation (displacement) values respectively for the i^{th} tube.

Here, a condensed summary of the model is provided for clarity and completeness. Homogeneous frames denoted as g(s) are propagated along the backbone of the robot. Here s denotes the arc-length. The local origin is located at r(s), and by convention, the local orientation R(s) is such that the local z-axis of the material frame aligns with the tangent vectors on an arc-length parametric curve that represents the CTR backbone.

$$g ( s ) := [ \begin{matrix}R ( s ) & r ( s ) \\ {0}^{⊤} & 1\end{matrix} ] \qquad ( 1 )$$

The Cosserat theory of slender rods is used to derive the set of differential equations governing the shape of the CTR's backbone. For conciseness of notation, the arc-length argument s is omitted in the following equations:

$${g}^{′} = g \hat{ξ} \qquad ( 2 )$$

$${θ}^{′} = {u}_{i , z} - {u}_{1 , z} i = 2 , … , n$$

$${u}_{i , z}^{′} = \frac{{E}_{i} {I}_{i}}{{G}_{i} {J}_{i}} ( {u}_{i , x} {u}_{i , y}^{*} - {u}_{i , y} {u}_{i , x}^{*} ) i = 1 , … , n$$

$${m}_{x , y}^{′} = ( - \hat{u} m - {e}_{3} {R}_{1}^{⊤} {F}_{ext} ) {\vert}_{x , y}$$

where Eᵢ, Gᵢ, Iᵢ and Jᵢ denote the Young's modulus and shear modulus, second moment of area, and polar moment of inertia of the tube's cross-section, respectively. The \hat{ } is the operator that converts R³ to SO(3), where so(3) is the Lie algebra of SO(3). The pre-curvature of the i^{th} tube is uᵢ^{*} and the axial angle θᵢ defines the angle relative to tube one (taken as reference). The externally applied point force at the distal end of the CTR is Fₑₓₜ. Furthermore the auxiliary variables u_{ix,y}, uᵢ, m_{z}, and ξ are expressed in terms of state variables, as

$${R}_{θ i} = [ \begin{matrix}cos ( {θ}_{i} ) & - sin ( {θ}_{i} ) & 0 \\ sin ( {θ}_{i} ) & cos ( {θ}_{i} ) & 0 \\ 0 & 0 & 1\end{matrix} ] \qquad ( 3 )$$

$${u}_{{1}_{x , y}} = {( {\sum }_{i = 1}^{n} {K}_{i} )}^{- 1} ( {m}_{x , y} + ( {\sum }_{i = 1}^{m} {R}_{{θ}_{i}} {K}_{i} {u}_{i}^{*} ) {\vert}_{x , y} )$$

$${u}_{i} = {R}_{{θ}_{i}}^{⊤} {u}_{1} + {θ}_{1}^{′} {e}_{3}$$

$${m}_{z} = {\sum }_{i = 1}^{n} {G}_{i} {J}_{i} {u}_{i , z}$$

$$ξ = {[ 001 {u}_{1}^{⊤} ]}^{⊤}$$

### Boundary Conditions

The differential equations in (2) can then be solved subject to appropriate boundary conditions on R, r, θᵢ, and m_{x,y}. Namely, at the proximal end (s=0), the initial pose of the robot and the initial torsional twist of the tubes1 can be expressed as

$$r ( 0 ) = {[ 000 ]}^{⊤} \qquad ( 4 )$$

$$R ( 0 ) = [ \begin{matrix}cos ( {α}_{1} - {β}_{1} {u}_{1 , z} ) & - sin ( {α}_{1} - {β}_{1} {u}_{1 , z} ) & 0 \\ sin ( {α}_{1} - {β}_{1} {u}_{1 , z} ) & cos ( {α}_{1} - {β}_{1} {u}_{1 , z} ) & 0 \\ 0 & 0 & 1\end{matrix} ]$$

$${θ}_{i} = ( {α}_{i} - {β}_{i} {u}_{i , z} ) - ( {α}_{1} - {β}_{1} {u}_{1 , z} ) , i = 1 , … , n$$

At the distal end of each tube (s= [symbol] i), it is necessary for the rate of torsional twist in the tubes to disappear. Additionally, if an external moment load is applied at the distal end of the innermost tube (s= [symbol] 1), the total sum of moments must also be zero, i.e.,

$${G}_{1} {J}_{1} {u}_{1 , z} ( {l}_{1} ) = {e}_{3}^{⊤} {R}_{1}^{⊤} {M}_{ext} \qquad ( 5 )$$

$${G}_{1} {J}_{1} {u}_{1 , z} ( {l}_{1} ) = 0 , i = 2 , … , n$$

$${m}_{x , y} ( {l}_{1} ) = ( {R}_{1}^{⊤} {M}_{ext} ) {\vert}_{x , y}$$

Lastly, at each arc-length where any tube in the CTR assembly ends or its pre-curvature transitions from a straight to a curved section, one must ensure the continuity of the bending moments, local positions, and orientations of tube across the transition points (split boundaries), i.e.,

$${g}_{i} ( {s}^{-} ) = {g}_{i} ( {s}^{+} ) \qquad ( 6 )$$

$${m}_{i} ( {s}^{-} ) = {m}_{i} ( {s}^{+} )$$

where the superscripts +, − denote the value of the variables immediately to the left and right side of the said transition points.

TABLE III
Translational actuator configurations for calibration.

| | |
|---|---|
| Actuator | Actuation value [mm] |

| | | | | | |
|---|---|---|---|---|---|
| β₁ | −131.0 | −111.0 | −91.0 | −68.0 | −55.0 |
| β₂ | −61.0 | −61.0 | −50.0 | −38.0 | −25.0 |
| β₃ | 0.0 | 0.0 | 0.0 | 0.0 | 0.0 |

TABLE IV
Calibrated tube parameters

| | Tube 1 | Tube 2 | Tube 3 |
|---|---|---|---|
| | (innermost) | (intermediate) | (outermost) |
| Young's Modulus | 50.00 | 179.808 | 289.295 |
| (E) [GPa] | | | |
| Shear Modulus | 58.9514 | 399.993 | — |
| (G) [GPa] | | | |

### Model Calibration

Given the computational complexity of the kinematic model discussed in this section, the model was implemented in C++ using an explicit forward integration scheme with an eighth-order adaptive Adams-Bashforth-Moulton integration method available in the Boost libraries. This improves the computation speed and enables real-time performance.

To define the mechanical properties of the tubes outlined in Table II, a calibration procedure has also been developed using a variant of the Nelder-Mead algorithm available with the NLopt library. This involves solving a box-constrained optimization problem over the vector of parameters p=[E₁, G₁, E₂, G₂, E₃]. Namely, for a robot configuration qᵢ and a set of parameters p∈P, if the distal end of the robot as predicted by the model x^{m}(qᵢ, p) and as measured by the EM sensors x^{e}(qᵢ), model calibration is achieved upon determining the optimal value p* that solves

$${p}^{*} = {arg min}_{p ∈ P} {\sum }_{i = 1}^{60} \{ ‖ {x}^{m} ( {q}_{i} , p ) - {x}^{e} ( {q}_{i} ) ‖ \} \qquad ( 7 )$$

$$s . t . \qquad 30 GPa \leq E \leq 400 GPa$$

$$30 GPa \leq G \leq 400 GPa$$

Specifically, the optimization problem (7) is solved for the set of configurations resulting from actuating the robot to the five linear translational positions in Table II and, at each of these translational positions, rotate the inner tube by twelve π/6 radian increments. The calibrated values for the parameters resulting from this procedure can be see in in Table IV.

Upon completion of the calibration, the updated C++ model, now with the newly calibrated kinematic parameters, yielded a mean prediction open-loop error of 7.56=6.50 mm for the set of linear actuations listed above. In the worst-case, the largest prediction error by the calibrated model was determined to be 4.54% of the overall length of the backbone, which is consistent with accuracy levels reported in previous studies.

### Closed-Loop Position Control

### Resolved Motion Rate Inverse Kinematics

In this section, the closed-loop, quasi-static position control of the hand-held CTR is implemented. The approach involves the implementation of a resolved-motion rate inverse kinematics-based strategy to address the task-space position control problem for the manipulator. Furthermore, by utilizing the inherent redundancy in these manipulators, self-motion capabilities of the robot are explored to achieve an additional objective, namely, collision avoidance for the linear translational joints of the hand-held CTR.

The velocity kinematics for the CTR are obtained by taking the time derivatevs of the kinematic model, and are classically given by

$$\dot{x} = J ( q ) \dot{q} \qquad ( 8 )$$

where \dot{x}(t)∈R^{m} denotes the task-space velocities, \dot{q}(t)∈Rⁿ the joint-space velocities, and J(·) ★R^{m×n} the finite difference Jacobian matrix whose i^{th} column is computed as

$$\begin{matrix}{J}_{i} \approx {( \frac{{g}_{i} ( s ) - g ( s )}{Δ {q}_{i}} )}^{⋁} & i = 1 , … , n\end{matrix} \qquad ( 9 )$$

where gᵢ(s) is the solution of the BVP in (2) when qᵢ is perturbed by Δqᵢ, and ^{∨} is the inverse of the ^{∧} operator.

As the number of joints n exceed the rank of the Jacobian matrix, owing to the kinematic redundancy of the CTR, the solution to the inverse kinematics problem can be decomposed into

$$\dot{q} = {\dot{q}}_{P} + {\dot{q}}_{N} \qquad ( 10 )$$

where \dot{q}ₚ∈Rⁿ represents the minimum norm, particular solution and \dot{q}_{N}∈Rⁿ represents the homogeneous solution given by

$${\dot{q}}_{P} = {J}^{\#} \dot{x} \qquad ( 11 )$$

$${\dot{q}}_{N} = {I}_{n} - {J}^{\#} J ( κ \frac{\partial h ( q )}{\partial q} ) \qquad ( 12 )$$

where J^{#} is the Moore-Penrose pseudoinverse of the Jacobian matrix and q_{N} is referred to as the self-motion of the robot, as J\dot{q}_{N}∈N (J). Through a local gradient projection of a performance criterion h(q) on the kernel of the Jacobian, bounds on the joint velocities of the manipulator are imposed as secondary goals in order to avoid collisions between the linear actuators of the hand-held CTR. In this implementation, the joint velocity criterion has been adopted for κ<0:

$$h ( q ) = {\sum }_{i = 1}^{n} \frac{1}{4} \frac{{( {q}_{i , max} - {q}_{i , min} )}^{2}}{( {q}_{i , max} - q ) ( q - {q}_{i , min} )} \qquad ( 13 )$$

### EM Filtering Integration

The effectiveness of the position feedback loop in the control framework is contingent on the quality of the readings of the EM tracking sensors, which are prone to ambient noise and interference from ferromagnetic sources. A Kalman filtering approach has been implemented, employing a nearly constant velocity model, for the EM sensor data to improve the reliability and precision of the position estimation.

It is known that the performance of the Kalman filter does not depend separately on the variances of the process and measurement noise, but rather on their ratio [43]. In view of this, these variances are estimated individually.

First, the statistics of the EM measurement noise were determined empirically in a procedural experiment which involved moving the sensor to different regions within the operating volume of the tracking system while collecting data. The measurement noise covariance matrix for the filter was then determined from this preliminary set of N sensor measurements γᵢ by

$$R = \frac{1}{N - 1} {\sum }_{i = 1}^{N} ( {y}_{i} - {\overline{y}}_{i} ) {( {y}_{i} - {\overline{y}}_{i} )}^{T} \qquad ( 14 )$$

For estimating the process covariance matrix Q, from the outset, a diagonal structure

$$Q = {σ}_{e}^{2} I$$

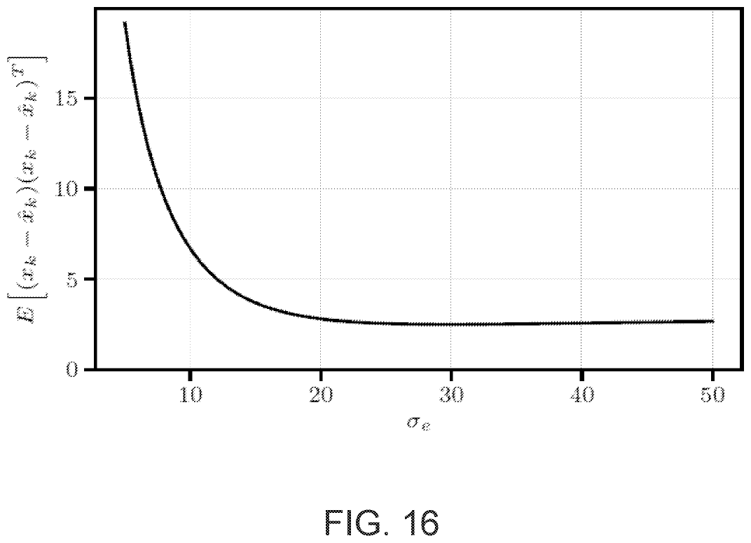

known ground truth trajectory and, subsequently, run a Monte-Carlo filtering simulation where a line search on the parameter σₑ produces the minimum mean square estimation error with respect to the ground truth as in (15). From this procedure, and as can be seen in the asymptotic behavior in FIG. 16, the process noise variance

$$Q = {σ}_{e}^{2} {I}_{6 \times 6}$$

was parameterized by σₑ=25.00 as the variance that minimizes the mean-square error of the filter estimates, i.e.,

$${min}_{Q} \{ \frac{1}{N} {\sum }_{k = 1}^{N} ( {x}_{k} - {\hat{x}}_{k} ) {( {x}_{k} - {\hat{x}}_{k} )}^{T} \} \qquad ( 15 )$$

$$\begin{matrix}s . t . & 5. \leq {σ}_{e} \leq 50.0\end{matrix}$$

### Control Experiments

The control experiments were conducted in two ways. First, a free space control with the robot attached to a passive arm (see FIG. 8A) to validate the control system performance without external disturbances. Second, when the robot was manually grasped and the tube was inserted into an abdominal phantom (see FIG. 8B) to validate the control system performance in a more clinically realistic scenario. In both experiments, an EM tracking system was used to localize the robot's body and the CTR tip.

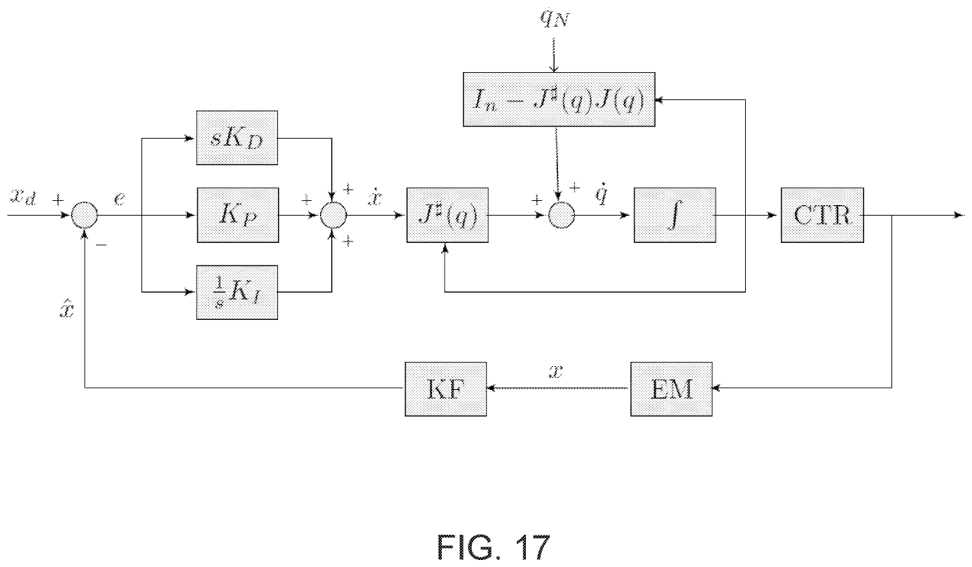

The task-space controller runs on a Linux-based computer with a 3.3 GHz core i9-10940X Intel processor and implements a C++ version of the resolved motion rate inverse kinematics described in Section V-A. The overall control framework includes a proportional-integral-derivative (PID) controller and utilizes Kalman filtering of the EM sensor signals, as shown in the control block diagram of FIG. 17.

From this background, the control experiments aimed to tackle the trajectory tracking problem for three different trajectories in task-space, each characterized by varying levels of complexity. Helical, square, and hypocycloidal trajectories were considered. The reference trajectories were discretized into 200 equally spaced 3D points describing a path p(t)=(x(t), y(t), z(t)) updated to the controller at each time interval t∈[0, 180] seconds. Additionally, a section of a trajectory generated using pre-operative imaging of a PCNL case for accessing the renal stones from the skin surface, generated in prior studies, was utilized to validate the system's performance in a realistic scenario. In all experiments, the trajectories were defined in the EM tracking frame and were transferred to the robot frame on the fly for closed-loop con-trol. Therefore, in experiments where the robot was manually grasped, the controller needed to compensate for the user's hand motion.

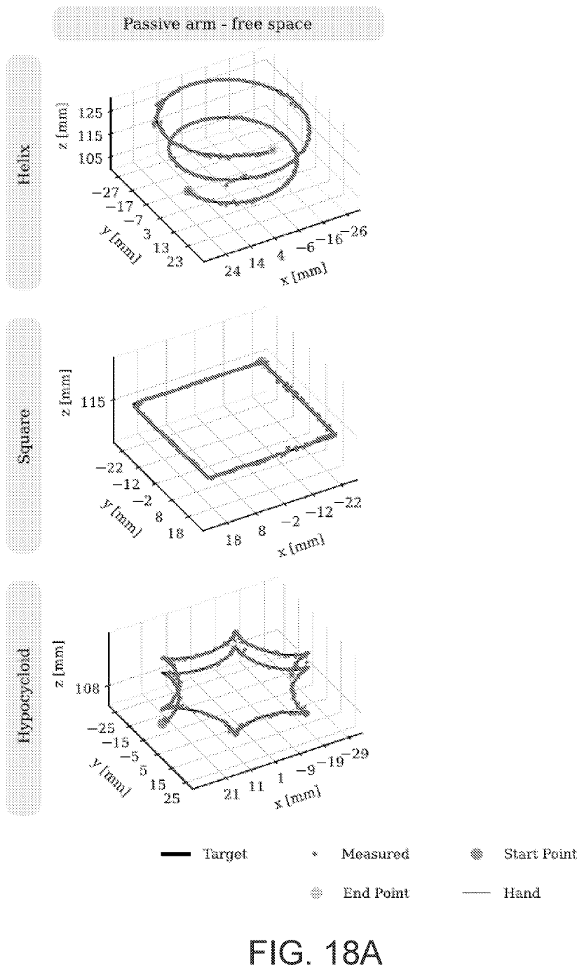

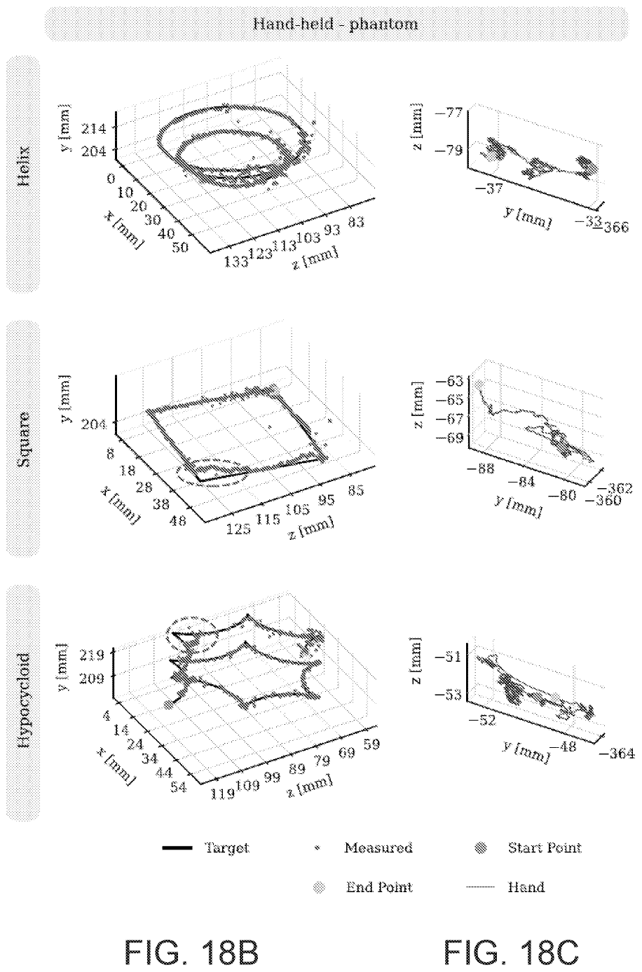

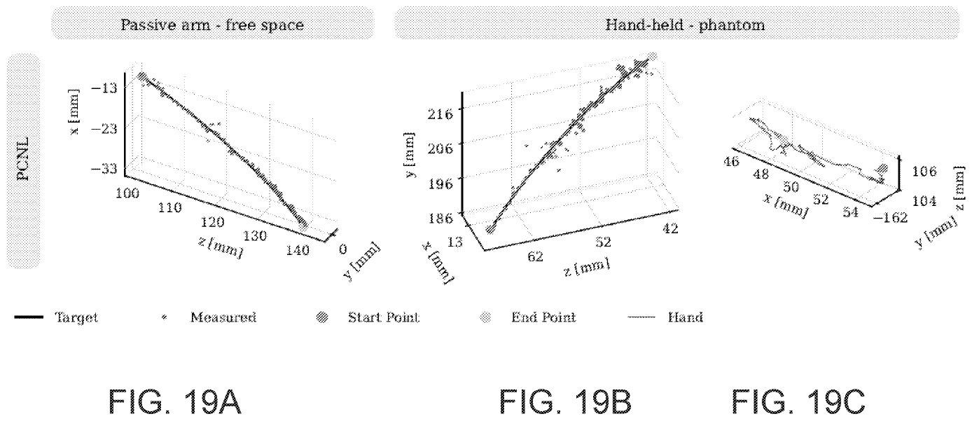

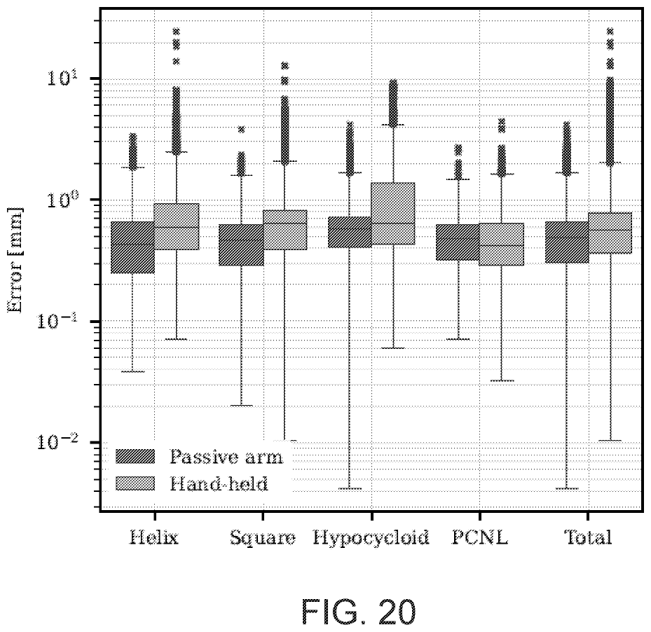

The plots in FIG. 18A-18C show both the ground truth and the CTR end-effector positions under closed-loop control at each of the 200 discretized points along the helical, square, and hypocycloidal trajectories when the robot was attached to a passive arm and when manually grasped. To assess performance, the distribution of the position errors for each one of the trajectories under closed-loop control is shown in the boxplots in FIG. 20. Using a passive arm, mean position errors of 0.55±0.47 mm, 0.50±0.32 mm, and 0.70±0.52 mm were observed for the helical, square, and hypocycloidal trajectory, respectively. The associated mean position errors when the robot was manually grasped were slightly higher at 0.84±0.95 mm, 0.90±0.96 mm, and 1.29±1.62 mm for the helical, square, and hypocycloidal trajectory, respectively.

It should be noted that the position tracking error during manual grasping is influenced by the operator's hand motions, resulting in higher error margins. Substantial hand motion can cause the target point in the robot's frame to deviate from the robot's workspace, rendering the target unreachable. Some of these unreachable scenarios are indicated by dashed blue lines in FIG. 18B.

The plots in FIG. 18A-18C show the results for the trajectory generated from the clinical case. For this specific trajectory, the observed position tracking error was 0.48±0.22 mm, and 0.48±0.32 mm for the passive arm-supported and hand-grasped scenarios, respectively. It can be seen that the controller could compensate for the operator's hand motion of around 10 mm while tracking the prescribed trajectory, as shown in the hand motion diagram in FIG. 18A-18C. It should be noted that the implementation of the proposed controller led to a substantial decrease in the open-loop mean error from 7.56-6.50 mm to the aforementioned minimal closed-loop errors.

The first hand-held CTR was introduced specifically designed to be used as an assistive tool in PCNL. The novel mechanical design features a slightly oval cross-section, with compact dimensions (322 mm in length, 46 mm along its maximum cross-sectional semi-axis), and an overall weight of 570 g. These design choices yield a lightweight system that ergonomically conforms to the operator's hand, facilitating ease of hand manipulation while mitigating muscle fatigue. To investigate the interactions between the hand-held CTR and an operator, an ergonomic analysis was conducted. The analysis focused on the surface sEMG activation signals of arm muscles for 6 urologists and 5 non-clinicians while manipulating the hand-held CTR in various poses simulating those of a urologist treating lower, mid, and upper pole renal stones, with the patient positioned both supine and prone.

The findings indicated that the physical strain experienced by the operator while manually manipulating the CTR was less than that observed when operators manipulated the LithoClast device, a commonly used ultrasonic and pneumatic lithotripter for ablating renal stones, and was used in the experiments as a comparative benchmark. A statistical t-test, conducted at a significance level of α=0.01, on the sEMG muscle activation signals, demonstrated significantly lower muscle activation levels for the group of 6 urologists when using the hand-held CTR compared to the commercially available LithoClast.

Furthermore, the hand-held CTR was evaluated under closed-loop control by analyzing its trajectory tracking capabilities along four distinct trajectories in task-space, namely helical, square, hypocycloidal and a trajectory for renal stone access from the kidney surface generated from the preoperative CT images of a patient who underwent PCNL. The mean positioning accuracies for the robot while tracking these trajec-tories were computed to be at 0.84-0.95 mm, 0.90±0.96 mm, 1.29±1.62 mm, and 0.48±0.32 mm respectively. These results indicate that the hand-held CTR exhibits closed-loop performance levels in line with the specific requirements of PCNL procedures for which it has been designed.

### Example 2

Linear and/or Nonlinear Control of the Hand-Held ACR Device

Multiple control strategies have been developed for the hand-held ACR system. One non-limiting example of a control software is currently being developed under the Robot Operating System (ROS) and interfaced with a navigation software, using a network protocol. The following are non-limiting examples of use or control protocols.

### i) Autonomous Image-Guided Control for the ACR to Follow a Pre-Defined Path to Reach the Target:

In this phase, the surgeon inserts the ACR into the body while the lateral motion of the ACR is autonomously controlled by a computer to follow the desired trajectory from the point of insertion of the device to the target location. The desired trajectory could be automatically generated or manually delineated by an expert clinician on diagnostic imaging. The tracking error will be the difference in the tip position and orientation measured by the EM sensor and the pre-defined trajectory at the corresponding point for the same insertion length. These errors will be automatically corrected by the computer algorithm to ensure that the error is always within the acceptable range. A kinematics controller based on the augmented Jacobian is used to control the robot to minimize position and orientation errors in real-time and thereby follow the desired trajectory.

In another embodiment, the desired trajectory for the ACR will be generated preoperatively using the diagnostic imaging data and image processing algorithms. Thereafter, the autonomous controller for the ACR will control the insertion and rotation of the tubes to following the desired trajectory from the skin puncture location toward specific target sites within the kidney while maintaining a constraint at the calyx. In this non-limiting example, the ACR's deployment will occur in a quasi follow-the-leader fashion where its backbone approximately follows the trajectory traced by the distal end while adhering to certain constraints such as avoiding sensitive anatomical structures and passing through a predetermined renal calyx en route to reaching its intended distal targets within the kidney to cover the entire stone burden.

### ii) Manual Control Under Direct Visualization to Allow Greater Manipulation of the ACR Device:

The ACR can also be switched to manual control mode so that the clinician can control the tip position of the robot using the 2-DOF control knobs and the two buttons on the handle of the device. A control scheme that commands the ACR to follow trajectories generated in real-time are sent from a control console. The clinician will use the control knobs and buttons to command the motions of the ACR under direct visual feedback or under imaging feedback such as ultrasound, CT or MRI. Three DOFs are needed for controlling the tip position but there are 4 DOFs in the device. The additional one-DOF redundancy will be used for changing the pitch or yaw angle of the ACR so that the tip of the ACR can be oriented appropriately to ensure optimal delivery of therapy.

### Navigation System

In a non-limiting example, the hand-held ACR system is integrated with an intraoperative navigation system to precisely guide the device under real-time imaging or context-specific diagnostic imaging registered to the coordinates of the ACR device. A visual interface for the ACR in a patient-specific anatomical map provides a global view of the ACR. The navigation system provides either a 2D reformatted display of the CT or MRI in the coordinate frame of the ACR device or 3D immersive display with augmented reality options. Setting up the navigation system in 3D could include the following three main steps: (1) segment and create 3D models of key anatomical structures from preoperative CT or MRI imaging; (2) develop a registration algorithm to register the electromagnetic tracking system to the preoperative diagnostic imaging; (3) refine the result of the static registration algorithm by developing a dynamic registration approach using implantable fiducials or intraoperative imaging such as ultrasound or C-arm CT imaging to further refine the registration from the previous step. Further, the ACR will be displayed to the user within the anatomical map in three dimensions in the patient-specific anatomical map. The navigation map will help localize the target, thereby improving the navigation of the ACR to reach the target while avoiding the critical structures.

### Patient-Specific Design

Based on preoperative imaging data, the ACR tool can be customized to cater to individual patient anatomical requirements and stone burden characteristics. The active cannula is remarkably prolific with regard to kinematic tube parameters and, consequently, kinematic capabilities. This notably large design space, namely the set of parameters governing the kinematics of the robotic device (tubes' diameters, stiffness, inner and outer radii, lengths of the curved and straight segments, and radius of curvature of the curved section) allows for a choice of tube parameters that can be optimized for each patient's specific needs. A non-limiting possible method to address this issue involves approximating the renal stones' shape to a mathematically simple geometry, specifically an ellipsoid. Following that, the design of the anatomically constrained cannula can be optimized to improve kinematic ability along the principal directions of the stone burden as given by the ellipsoidal approximation of the renal calculi.

### Example 3

### Follow-the-Leader (FTL) Deployment

In this approach, a flexible robot is guided along a carefully prescribed piecewise constant-curvature trajectory path, ensuring that its slender body (backbone) remains undeflected while tracing the same route traversed by its distal-end (tip). In a perfect FTL deployment sequence, the volume displaced by the robot upon insertion into the body equals the volume of its own sheath.

Mathematically speaking, the shape of the robot is described by a time-varying arc-length parameterized transformation g(s,t) which assigns a position p(s, t)∈R³ and an orientation R(s, t)∈SE(3) to each arc-length s∈[0,L] along the centerline of the tubes.

For a perfect FTL, it is required that the position of the robot's centerline at any arc-length does not change in time, i.e.,

$$\dot{p} ( s , t ) := \frac{\partial p ( s , t )}{\partial t} = 0$$

The above condition entails a constraint on the admissible values of the backbone curvature u(s,t) in a follow-the-leader deployment. That is to say, since the arc-length parameterized space curve that models the robot's centerline is not allowed to change, save in its tangential direction to allow growth/contraction (change in length) in the domain s∈[0,L.].

In this respect, and in a similar vein, another way to approach the FTL deployment problem is to frame it based on the robot's local curvature.

In the absence of any external loads and axial torsion of the tubes, the quasistatic local curvature of a n-tube CTR is

$$u ( s , t ) = {[ {\sum }_{i = 1}^{n} {K}_{i} ]}^{- 1} {\sum }_{i = 1}^{n} R ( {θ}_{i} ( t ) ) {K}_{i} {u}_{i}^{*} ( s - {h}_{i} ( t ) )$$

where hᵢ is defined as the distance between the distal end of the robot and that of the i^{th} tube. The stiffness of the i^{th} tube is Kᵢ and its precurvature is

$${u}_{i}^{*} ( s ) .$$

In light of the above equation, for FTL deployment, the backbone curvature of the robot must remain constant along the entire arc length. Considering that the curvature of the robot can be expressed in terms of arc-length s and time t, the FTL condition in terms of curvature can be stated as:

$$\frac{\partial u ( s , t )}{\partial t} + v \frac{\partial u ( s , t )}{\partial s} = 0 \qquad ∀ s , t$$

The above equation indicates that for a robot to be deployed in a FTL fashion, the local curvature of each cross-section defined by an arc-length s must pass to the immediate adjacent cross-section with insertion velocity v toward the distal end of the robot.

In a non-limiting example, and in a similar way as briefly described above, the present steerable cannula will be deployed in a near follow-the-leader manner. Through a sequence of meticulously planned actuation commands, the cannula's motion is guided so that its backbone navigates, as closely as possible, a desired trajectory in a snake-like fashion where the curved shape of the robot remains unchanged as the tip progresses forward. This methodology empowers the cannula to traverse through confined spaces and intricate anatomical structures, breaching the fascia while adhering to anatomical constraints. As a result, it facilitates precise and controlled movements within the human body, safeguarding the integrity of sensitive surrounding anatomical structures.

1. An active cannula robot system, the system comprising:
a housing with a proximal end and a distal end, including:
a first cannula unit, including:
a first motor and a second motor positioned proximally in the housing;
a carriage unit operably coupled and positioned distally to the first motor and the second motor within the housing;
an active cannula tube extending longitudinally between and beyond the proximal end and distal end and configured to be actuated by the carriage unit;
a stationary cannula tube fixed to an outer surface of the distal end of the housing and extending distally; and
a processor configured to control the active cannula robot system.

2. The system of claim 1, wherein the first motor produces an axial rotation in the active cannula tube and the second motor produces a longitudinal translation of the active cannula tube.

3. The system of claim 1, wherein the active cannula tube includes a straight section extending through the housing and a curved section in a distal end of the active cannula tube that is external to the distal end of the housing.

4. The system of claim 1, wherein the carriage unit includes:
a rotation unit including:
a splined shaft coupled to the first motor and extending to the distal end of the housing;
a first gear axially coupled around the splined shaft; and
a second gear coupled to a shaft collar fixed to the active cannula tube and configured to engage the first gear; and
a translation unit including:
a lead screw coupled to the second motor and extending to the distal end of the housing; and
a linker coupled to the rotation unit and translation unit.

5. The system of claim 1, wherein the housing further includes a second cannula unit, wherein one cannula tube is concentrically arranged relative to another cannula tube.

6. The system of claim 1 or 5, wherein the active cannula tube is concentrically positioned within the stationary cannula tube.

7. The system of claim 5, wherein each of the first and second cannula units are operated separately.

8. The system of claim 5, wherein the housing further includes a printed circuit board (PCB) positioned proximally to the first motor and the second motor of each of the first cannula unit and the second cannula unit within the housing, and operably connected to the first cannula unit and the second cannula unit, the processor, and a power supply.

9. The system of claim 1, wherein the active cannula tube or stationary tube are flexible relative to the housing.

10. The system of claim 1, wherein the active cannula tube or stationary tube includes nitinol.

11. The system of claim 1, further comprising controls arranged to receive user input via a single hand and communicate the user input to the processor to control the active cannula robot system.

12. The system of claim 1, wherein the housing includes a force sensor coupled to the distal end.

13. The system of claim 1, wherein the housing includes an electromagnetic (EM) sensor coupled to a surface of the housing.

14. The system of claim 13, wherein the active cannula tube includes a distal EM sensor at its distal end.

15. The system of claim 14, further comprising an EM tracker configured to track at least one of the EM sensor or the distal EM sensor.

16. The system of claim 1, wherein the processor includes an navigation system configured to guide the active cannula robot system to a target location inside a patient.

17. An active cannula robot system, the system comprising:
a housing with a proximal end and a distal end, wherein the proximal end and distal end are reversibly separable into a proximal portion and a distal portion, the housing including:
the proximal portion including:
one or more pairs of motors, and
a first coupling operably connected and distally positioned to each motor in each of the one or more pairs of motors;
the distal portion including:
one or more carriage units corresponding to a number of the one or more pairs of motors,
two second couplings operably connected and proximally positioned to each carriage unit, wherein each of the first couplings and each of the second couplings reversibly connect to provide a mechanical and an electrical connection between each of the one or more pairs of motors and the one or more carriage units,
a stationary cannula tube fixed to an outer surface of the distal end and extending distally; and
a channel coupled to the outer surface of the distal portion;
an active cannula tube configured to be actuated by the one or more carriage units and introduced into the distal portion by the channel and extend longitudinally therein and distally outside the distal end; and
a processor configured to control the active cannula robot system.

18. The system of claim 17, wherein the distal portion further includes a reference electromagnetic (EM) sensor coupled to the housing.

19. The system of claim 18, wherein the active cannula tube includes a distal EM sensor at its distal end.

20. The system of claim 19, further comprising an EM tracker configured to track at least one of the EM sensor or the distal EM sensor.

21. The system of claim 17, wherein the proximal portion further includes a printed circuit board (PCB) positioned proximally to the one or more pairs of motors within the housing, and operably connected to the one or more pairs of motors, the processor, and a power supply.

22. A method of intraoperative navigation of an active cannula robot (ACR), the method including the steps of:
acquiring pre-operative medical imaging data;
applying image segmentation to the medical imaging data;
developing a three-dimensional model of one or more structures in the segmented medical image data;
developing a registration algorithm of an electromagnetic tracking system, wherein the electromagnetic tracking system is configured to monitor a position of a distal tip of the ACR; and
registering the electromagnetic tracking system to the pre-operative medical image data.

23. The method of claim 22, wherein the pre-operative medical imaging data is one of magnetic resonance imaging (MRI) data or computed tomography (CT) imaging data.
